(19) United States
(12) Patent Application Publication
(10) Pub. No.: US 2023/0350931 A1
(43) Pub. Date: Nov. 2, 2023

(54) SYSTEM OF SEARCHING AND FILTERING ENTITIES

(71) Applicant: BenevolentAI Technology Limited, London (GB)

(72) Inventors: Neal Ryan Lewis, Brooklyn, NY (US); Oliver Oechsle, London (GB)

(73) Assignee: BenevolentAI Technology Limited, London (GB)

(21) Appl. No.: 17/786,909

(22) PCT Filed: Dec. 11, 2020
(86) PCT No.: PCT/GB2020/053176

§ 371 Date: Jun. 17, 2022

Related U.S. Application Data
(60) Provisional application No. 62/951,557, filed on Dec. 20, 2019.

(51) Int. Cl. G06F 16/33; G06F 16/36; G06F 16/338

(52) U.S. Cl. CPC G06F 16/3338; G06F 16/367; G06F 16/338

(57) ABSTRACT

Methods, apparatus, system and computer-implemented method(s) are provided for creating a graph of entities of interest and relationships thereto. A search query is received corresponding to entities of interest. The search query including data representative of a first set of entities. An expanded search query is generated based on inputting the received search query to one or more entity expansion process(es) or engine(s). The expanded search query including data representative of a second set of entities and the first set of entities. Creating a graph of entities of interest and relationships thereto based on processing the expanded search query with data representative of a corpus of text. Creating the graph by processing the expanded search query to filter an existing graph of entities of interest and relationships thereto based on the expanded search query. The existing graph of entities of interest and relationships thereto is previously generated based on the corpus of text.

The present application relates to a system and method for lexicon expansion for generating a graph of entities and relationships thereto from a corpus of text.

### BACKGROUND

Sheer volume of data in a particular field or sub field of technology or research area makes individually reading each piece of new data (i.e. background/literature/text) difficult or time-consuming if not impossible for a researcher to keep up with, let alone having to analyse and derive meaningful correlations from the data. Given that more and more data is being generated, manual efforts by each researcher alone becomes insufficient to tackle the increased volume of data, which grows day by day. As such, although there are numerous methods of using computers to automate and/or assess this increased volume of data, extract pertinent information such as relevant documents, and/or relevant information within documents for each different researcher and/or different topic/field of interest for a researcher remains difficult, and even intractable.

For example, document search engines are available for searching through a corpus of text and/or documents based on taking a search query from a user. Various search engine algorithms may search a search index based on the search query and output a plethora of tabulated results associated with the query. These results may still be intractable for a user and/or researcher to determine which are relevant, which to discard and which may lead to the next breakthrough or ground-breaking discovery. A lot of time is still spent by the user in curating and/or refining the result set.

There is indeed a need for such an invention that allows the creation of enhanced search results, expanding search query concepts for capturing the most relevant data and/or documents in any particular field, for example, such as biological and/or chemical sciences and providing an enhanced search result set that enables a user to systematically examine the search concepts according to their underlying relationships.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides a system for iteratively processing and expanding a search query to include relevant entities of interest, concepts of interest, words of interest, phrases of interest and the like to enhance a search of a corpus of text associated with the search query. The search query may include a first set of entity terms, phrases, words, or concepts of interest, which are processed using a corpus of text and/or multiple expansion process(es) based on, without limitation, for example machine learning models, database searches, graph based searches/traverses, which feedback expanded search terms for incorporation into the search query after validation. Once the search query has been sufficiently expanded to provide a robust search, it is used for searching a corpus of text and providing or building a graph from entities and/or relationships extracted by search. The corpus of text may also be represented as an entity graph with relationship edges and the like. The resulting entity graph may be provided and/or displayed to a user as the search results. Alternatively or additionally, the entity graph may be used as a training set for training one or more ML model(s) and the like.

In a first aspect, the present disclosure provides a computer-implemented method of creating a graph of entities of interest and relationships thereto, the method comprising: receiving a search query corresponding to entities of interest, the search query comprising data representative of a first set of entities; generating an expanded search query based on inputting the received search query to one or more entity expansion process(es) the expanded search query comprising data representative of a second set of entities and the first set of entities; and creating a graph of entities of interest and relationships thereto based on processing the expanded search query with data representative of a corpus of text.

As an option, generating the expanded search query further comprising: sending data representative of the received search query to said one or more entity expansion process(es); receiving data representative of the second set of entities from said one or more entity expansion process(es); and building an expanded search query corresponding to entities of interest based on a selection of data representative of the second set of entities and the first set of entities in relation to the entities of interest.

As an option, generating the expanded search query further comprising iteratively generating the expanded search query by: sending data representative of a current search query to said one or more entity expansion process(es), wherein, in the first iteration the current search query is the received search query; receiving data representative of the second set of entities from said one or more entity expansion process(es) based on the current search query; and building an expanded search query corresponding to entities of interest based on a selection of data representative of the second set of entities and the first set of entities in relation to the entities of interest; and updating the current search query with the expanded search query in response to performing another iteration.

As another option, building an expanded search query further comprising: receiving feedback that one or more of the entities of interest of the expanded search query are valid; and updating the expanded search query to only include data representative of the valid entities of interest.

As an option, creating the graph by processing the expanded search query further comprising: performing a search for entities of interest and relationships thereto in the corpus of unstructured text based on the expanded search query; and forming the graph of entities of interest and relationships thereto based on search results output from said search.

As an option, creating the graph by processing the expanded search query further comprises filtering an existing graph of entities of interest and relationships thereto based on the expanded search query, wherein the existing graph of entities of interest and relationships thereto is previously generated based on the corpus of text.

As an option, the method further comprising: receiving data representative of an additional set of entities output from one of the entity expansion process(es) configured to retrieve the additional set of entities from a database lookup using data representative of the search query corresponding to entities of interest; and combining the additional set of entities with the second set of entities.

As an option, the method further comprising: receiving data representative of an additional set of entities output from one of the entity expansion process(es) configured to extract entities of interest from or filter an existing graph of entities of interest and relationships thereto based on data representative of the search query; and combining the additional set of entities with the second set of entities.

As an option, the method further comprising: receiving data representative of an additional set of entities output from one of the entity expansion process(es) configured to input data representative of the search query to an ML model trained for predicting or identifying entities of interest and relationships thereto from a corpus of text; and combining the additional set of entities with the second set of entities.

As an option, the method further comprising: receiving data representative of an additional set of entities output from one of the entity expansion process(es) configured to search a corpus of text based on data representative of the search query; and combining the additional set of entities with the second set of entities.

Optionally, receiving data representative of an additional set of entities output from one of the entity expansion process(es) configured to retrieve the additional set of entities from a lexicon dictionary associated with entities; and combining the additional set of entities with the second set of entities.

As an option, creating a graph of entities of interest and relationships thereto further comprising: receiving the expanded search query based on a set of entity concepts associated with one or more entities; retrieving a set of entities and relationships thereto from the corpus of text based on inputting data representative of the expanded search query to a search engine or process configured for identifying one or more entity(ies) and relationships thereto based on the received expanded search query and the corpus of text; and generating a graph of entities of interest and relationships thereto using the retrieved set of entities and relationships.

As an option, retrieving a set of entities and relationships thereto from the corpus of text further comprising: inputting the expanded search query to a document extraction engine or process configured for identifying portions of text from the corpus of text associated with the expanded search query; and outputting one or more identified portions of text from the corpus of text associated with the expanded search query.

Optionally, retrieving a set of entities and relationships thereto from the corpus of text further comprising: inputting identified portions of text from the corpus of text associated with the expanded search query to a relationship extraction engine or process configured for identifying or predicting one or more entity(ies) and relationship(s) thereto in relation to the identified portions of text associated with the expanded search query; and outputting the identified or predicted set of entity(ies) and relationship(s) thereto.

As an option, the portions of text comprise a set of relevant documents from the corpus of text that are determined relevant to the entity concepts of the expanded search query.

As an option, the search engine or process comprises one or more ML search model(s) configured for identifying, predicting, ranking and/or scoring the plurality of documents associated with the expanded search query for determining the set of relevant documents.

Optionally, the search engine or process includes one or more information retrieval algorithms associated with document frequency and/or document similarity for performing a document search.

As an option, wherein the relationship extraction engine or process comprises one or more ML extraction model(s) configured for identifying, predicting, ranking and/or scoring a set of entities and relationships thereto in relation to the identified portions of the set of relevant documents and the expanded search query.

Optionally, receiving the search query based on data representative of the first set of entities further comprising receiving data representative of a selected first set of entity concepts associated with one or more entities of interest from a user.

As an option, generating an expanded search query comprising data representative of a second set of entities and the first set of entities further comprising: expanding the first set of entity concepts based on an expansion engine or process configured to expand the first set of entity concepts into data representative of a further relevant set of entity concepts; and generating an expanded search query based on the first set of entity concepts and/or the further relevant set of entity concepts.

Optionally, expanding the first set of entity concepts further comprising iteratively expanding the first set of entity concepts by: expanding a current set of entity concepts based on an expansion engine or process configured to expand the current set of entity concepts into data representative of a further relevant set of entity concepts, wherein in the first iteration the current set of entity concepts is the first set of entity concepts; receiving feedback that one or more of the entity concepts from the current set of entity concepts and/or further relevant set of entity concepts are valid or of interest; generating an expanded set of entity concepts based on the validated or of interest entity concepts from the current set of entity concepts and/or further relevant set of entity concepts; replacing the current set of entity concepts with the expanded set of entity concepts; iteratively performing the steps of expanding the current set of entity concepts, receiving feedback, and generating an expanded set of entity concepts until a stopping criterion in relation to expanding the current set of entity concepts is reached; and generating an expanded search query based on the current set of entity concepts.

As an option, updating the expansion engine or process configured to expand a set of entity concepts into further relevant set of entity concepts based on the received feedback of valid or of interest entity concepts.

As an option, updating the expansion engine or process prior to generating the expanded set of entity concepts.

As an option, the expansion engine or process comprises one or more entity expansion process(es) from the group of: an entity expansion process configured to extract additional entities of interest from or filter an existing graph of entities of interest and relationships thereto based on data representative of a set of entity concepts; an entity expansion process configured to input data representative of a set of entity concepts to an ML model trained for predicting or identifying additional entities of interest and relationships thereto from a corpus of text; an entity expansion process configured to search for additional entities of interest from a corpus of text based on inputting data representative of a search query associated with a set of entity concepts to a search engine coupled to the corpus of text; an entity expansion process configured to retrieve additional entities of interest from a lexicon dictionary associated with a set of entity concepts; and any other entity expansion process configured to retrieve additional entities from a database, dictionary system and/or search engine and the like in relation to a set of entity concepts.

Optionally, creating a graph of entities of interest and relationships thereto further comprises: generating a graph based on the retrieved sets of entities and relationships thereto; and updating an existing graph associated with the one or more entities of interest based on the generated graph. As an option, creating a graph further comprises generating a graph based on the retrieved sets of entities and relationships thereto.

Optionally, a graph of entities of interest and relationships thereto comprises a graph structure comprising a plurality of nodes based on a set of entities, wherein each node of the graph structure represents an entity and edges between a pair of nodes correspond to a particular relationship between the entities represented by the pair of nodes.

As an option, generating the graph further comprising: inferring a relationship edge between a first node and a second node of the graph when a first relationship edge exists from the first node to another node of the graph, and a second relationship edge exists from the another node to the second node; and inserting an inferred relationship edge between the first node and second node of the graph.

Optionally, generating the graph further comprising: inferring, for each node of the plurality of nodes in the graph, a relationship edge between said each node and an other node of the graph when a relationship edge path exists from said each node via one or more further nodes to the other node; and inserting an inferred relationship edge between said each node and the other node of the graph. As an option, weighting each relationship edge between each pair of nodes of the graph based on detecting the number of common relationships between the entities of said each pair of nodes from the set of entities and relationships.

Optionally, retrieving a set of entities and relationships thereto from the corpus of text using one or more ML extraction model(s) further comprising: generating predictions based on the expanded search query using one or more machine learning, ML, model(s) configured for predicting from the corpus of text a set of entity pairs and relationships associated with a set of entities associated with the search query, each predicted entity pair comprising an entity of a first type and an entity of a second type having an associated relationship therebetween identified from the corpus of text; and outputting the set of entity pairs and relationships as the set of entities and relationships.

As an option, the data representative of the graph is used as input labelled training datasets for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like.

AS an option, an entity comprises entity data associated with an entity type from at least the group of: gene; disease; compound/drug; protein; chemical; organ; biology; biological part; or any other entity type associated with bioinformatics, chem(o)informatics, biology, biochemistry, chemistry, medicine, pharmacology, and/or any other field relevant to diagnostic, treatment, and/or drug discovery and the like.

Optionally, an entity concept is data representative of entity information and/or entities from one or more fields or domains from the group of: biology, biochemistry, chemistry, medicine, chem(o)informatics, bioinformatics, pharmacology, and/or any other field relevant to diagnostic, treatment, and/or drug discovery and the like.

In a second aspect, the present disclosure provides a search engine apparatus for searching and filtering entity results for entities of interest from an corpus of text, the search engine apparatus comprising: an input component configured to receive a search query based on set of entity concepts associated with one or more entities; an expansion component configured to expand the received search query into an expanded search query comprising at least the set of entity concepts and/or further relevant entity concepts associated with the set of entity concepts; a search processor component configured to retrieve a set of entities and relationships thereto from the corpus of text based on inputting the expanded search query to a search engine configured for identifying and/or predicting one or more entity(ies) and relationship(s) thereto based on the expanded search query and the corpus of text; an entity result filtering component configured generate a graph using the retrieved set of entities and relationships thereto.

As an option, the input component, expansion component, the search processor component and/or the entity result filtering component are configured to implement the computer-implemented method according to any one or more features, steps, process(es) and/or methods of the first aspect, combinations thereof, modifications thereto and/or as herein described.

In a second aspect, the present disclosure provides an apparatus comprising a processor unit, a memory unit and a communication interface, the processor unit connected to the memory unit and the communication unit, wherein the apparatus is configured to implement the computer-implemented method according to any one or more features, steps, process(es), and/or method(s) of the first aspect, combinations thereof, modifications thereto and/or as herein described.

In a third aspect, the present disclosure provides a system comprising: a user interface configured for receiving one or more entity concepts associated with entities of interest; a search engine apparatus configured according to any one or more features, steps, process(es), and/or method(s) of the second or first aspects, combinations thereof, modifications thereto and/or as herein described, the search engine apparatus connected to the user interface for receiving the one or more entity concepts; and a display interface configured for displaying the graph associated with the one or more entity concepts.

In a fourth aspect, the present disclosure provides a system comprising: a receiver component configured to receive a search query corresponding to entities of interest, the search query comprising data representative of a first set of entities; a search query expansion component configured to generate an expanded search query based on inputting the received search query to one or more entity expansion process or engine, the expanded search query comprising data representative of a second set of entities and the first set of entities; and a graph creation component configured to create a graph of entities of interest and relationships thereto based on processing the expanded search query with data representative of a corpus of text.

As an option, the receiver component, search query expansion component, and the graph creation component are configured to implement the computer-implemented method according to any one or more features, steps, process(es), and/or method(s) of the first aspect, combinations thereof, modifications thereto and/or as herein described.

In a fifth aspect, the present disclosure provides a computer-readable medium comprising code or computer instructions stored thereon, which when executed by a processor unit, causes the processor unit to perform the computer-implemented method according to any one or more features, steps, process(es), and/or method(s) of the first aspect, combinations thereof, modifications thereto and/or as herein described.

As an option, the computer-implemented invention of the first aspect, search engine apparatus of the second aspect, the system(s) of the third and/or fourth aspects, the corpus of text comprises a large-scale document repository including a plurality of documents associated with a plurality of entity concepts and/or entities of interest and/or entities of relevance. The corpus of text may be a corpus of unstructured, semi-structured and/or structured text.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

The features of each of the above aspects and/or embodiments may be combined as appropriate, as would be apparent to a skilled person, and may be combined with any of the aspects of the invention. Indeed, the order of the embodiments and the ordering and location of the preferable features is indicative only and has no bearing on the features themselves. It is intended for each of the preferable and/or optional features to be interchangeable and/or combinable with not only all of the aspect and embodiments, but also each of preferable features.

### BRIEF DESCRIPTION OF THE DRAWINGS

Embodiments of the invention will be described, by way of example, with reference to the following drawings, in which:

FIG. 1a is a flow diagram illustrating an example process for expanding a search query for creating a graph of entities of interest and relationships thereto from a corpus of text according to the invention;

FIG. 1b is a schematic diagram illustrating an example search system for expanding a search query and creating a graph of entities of interest based on the process of FIG. 1a according to the invention;

FIG. 1c is a flow diagram illustrating an example process for search query expansion based on the process and search system of FIGS. 1a and 1b according to the invention;

FIG. 1d is a schematic diagram illustrating an example of creating a graph based on filtering an existing graph of entities of interest and relationships thereto in relation to the expanded search query of FIG. 1a to 1c according to the invention;

FIG. 1e is a schematic diagram illustrating another example of creating a graph of entities of interest and relationships thereto in relation to the expanded search query of FIG. 1a to 1c according to the invention;

FIG. 2a is a schematic diagram illustrating another example search system for automatically expanding key terms of biological concepts of a search query and retrieving relevant documents from a document repository based on the search query according to the invention;

FIG. 2b is a schematic diagram illustrating an relationship extraction and knowledge graph generation system for extracting biological entities and associated relationships from relevant documents retrieved from FIG. 2a according to the invention;

FIG. 2c is a schematic diagram illustrating an relationship extraction and knowledge graph update system for extracting biological entities and associated relationships from relevant documents retrieved from FIG. 2a according to the invention;

FIG. 3 is a schematic diagram illustrating an example knowledge graph associated with concepts and corresponding relationships thereto according to the invention;

FIG. 4a is a schematic diagram illustrating an example search engine (e.g. ML search model) for use with FIG. 1a-3 according to the invention;

FIG. 4b is a schematic diagram illustrating an example relationship extraction/identification engine (e.g. ML model) for use with FIG. 1a-4a according to the invention;

FIG. 5a is a schematic diagram illustrating a further example search system according to the invention;

FIG. 5b is a flow diagram illustrating an example process for searching and filtering biological entities of interest from a corpus of text for use with the search systems of FIG. 1a-5a according to the invention;

FIG. 5c is a flow diagram illustrating another example process for expanding biological concept search query of FIG. 5a according to the invention;

FIG. 5d is a flow diagram illustrating an example process for searching for relevant documents from the corpus of text based on the search system and/or search query of FIGS. 5a-5c according to the invention;

FIG. 5e is a flow diagram illustrating an example process for processing the relevant documents of FIG. 5d for extracting biological entities and associated relationships for creating a graph of entities of interest and relationships thereto according to the invention;

FIG. 6a is a schematic diagram illustrating a computing system and device according to the invention;

FIG. 6b is a schematic diagram illustrating a system according to the invention; and

FIG. 6c is a schematic diagram illustration another system according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples. For the avoidance of any doubt, the features described in any embodiment are combinable with the features of any other embodiment and/or any embodiment is combinable with any other embodiment unless express statement to the contrary is provided herein. Simply put, the features described herein are not intended to be distinct or exclusive but rather complementary and/or interchangeable.

The present invention is related to a process and system for expanding a search query associated with entities of interest and/or relationships thereto and for creating a graph of entities of interest and relationships thereto extracted from a corpus of text based on the expanded search query. In particular, the process and system may iteratively expand the search query based on using machine learning (ML) techniques and/or rule-based technique(s)/systems in an automated/semi-automated manner. In conjunction with one or more other ML techniques or rule-based algorithm(s) as described herein to generate and update knowledge graphs and/or sub graphs associated with entities and relationships thereto based on the expanded search query. Furthermore, the entities and relationships thereto extracted from the corpus of text may include, without limitation, for example, processing the corpus of text based on the search query using one or more ML techniques and/or rule-based techniques for identifying and/or extracting relevant documents based on the expanded search query, from which one or more entities and relationships thereto may be extracted using a further one or more ML techniques and/or rule-based algorithm(s) and the like for extracting entities and relationships thereto based on the expanded search query. The resulting set of entities and relationships thereto may be processed for generating and/or updating knowledge graphs and/or sub-graphs, where each node is associated with an entity and each edge linking nodes is associated with relationships between corresponding entities.

For example, the process and system may adaptively learn from both specific and generic patterns and nuances associated with the feedback in relation to expanding a search query, in turn, characterising the at least the one or more entities of interest for one or more particular entity type(s) (e.g. biological entity of interest associated with an entity type of disease, gene, protein, target, drug etc.) and at least one or more relationship entities associated with the relationship. The iterative procedure performed by the process and system described herein robustly generates expanded search queries and generates/updates knowledge graphs with relevant entities/relationships. The iterative procedure effectively improves the accuracy of extracting pertinent and/or relevant information associated with a search query with minimal human intervention and outputs and/or displays enhanced search results in the form of a knowledge graph and/or subgraph thereof associated with the search query enhancing the search experience, where users do not need to trawl through tabulated results associated with entities and relationships thereto.

A corpus of text, data or large-scale dataset may comprise or represent any information, text or data from one or more data source(s), content source(s), content provider(s) and the like. The large-scale data set or corpus of data/text, herein referred to as a corpus of text, may include, by way of example only but is not limited to, unstructured data/text, one or more unstructured text, semi-structured text, partially structured text. a collection of documents of natural language text, documents with structured headings for which together with portions of unstructured text from the document, structured text that may be processed, documents, sections of documents, sentences and/or paragraphs of documents, tables, structured data/text, a body of text, articles, patents and/or patent applications, publications, literature, text, email, images and/or videos, or any other information or data that may contain a wealth of information corresponding to one or more entity(ies) of interest, entity type(s) of interest, and/or entity concepts of interest and the like. The data associated with the corpus of text may be generated by and/or stored with or by one or more sources, content sources/providers, or a plurality of sources (e.g. PubMed, MEDLINE, Wikipedia, US Patent Office databases, European Patent Office databases and/or any other patent data bases) and which may be used to form the corpus of text from which entities of interest, entity types and entity relationships may be identified and/or extracted and the like.

Portions of text of the corpus of text may comprise or represent, without limitation, for example sentences, paragraphs, sections or segments of documents or data and/or whole documents and/or data, which may be retrieved from the corpus of text and processed for identifying, detecting and/or extracting one or more entities and/or relationships thereto. A portion of text may describe one or more entity relationships associated with one or more entity(ies) and/or entity(ies) of interest. The portion of text may be processed to identify, detect and/or extract, by way of example only but not limited to, a) one or more entity(ies) of interest, each of which may be separable entities of interest; and b) one or more relationship entity(ies) that form and/or define the relationship associated with the one or more entity(ies) of interest, which may be separable.

Such large-scale datasets or corpus of data/text may include data or information from one or more data sources, where each data source may provide data representative of a plurality of unstructured and/or structured text/documents, documents, articles or literature and the like. Although most documents, articles or literature from publishers, content providers/sources have a particular document format/structure, for example, PubMed documents are stored as XML with information about authors, journal, publication date and the sections and paragraphs in the document, such documents may be considered to be part of the corpus of data/text. For simplicity, the large-scale dataset or corpus of data/text is described herein, by way of example only but is not limited to, as a corpus of text. Such large-scale datasets or corpus of data/text may include data or information from one or more data sources, where each data source may provide data representative of a plurality of unstructured and/or structured text/documents, documents, articles or literature and the like. Although most documents, articles or literature from publishers, content providers/sources have a particular document format/structure, for example, PubMed documents are stored as XML with information about authors, journal, publication date and the sections and paragraphs in the document, such documents are considered to be part of the corpus of data/text. For simplicity, the large-scale dataset or corpus of data/text is described herein, by way of example only but is not limited to, as a corpus of text.

ML techniques herein used may include but are not limited to neural network (NN) structures, tree/graph-based classifiers, linear models and the like and/or any ML technique suitable for modelling/operating on the set of embeddings and/or an embedding vocabulary dataset generated during the training of an ML model or classifier. The trained ML model or classifier may be used to extract entities/relationships from the text corpus or a portion of the text. The set of embeddings and/or an embedding vocabulary dataset are generated for each of one or more relationship entity(ies) (e.g. specific relationship entities found in the portion of text describing a relationship associated with one or more specific biological entity(ies) of interest) with respect to the use of the ML techniques.

ML technique(s) may further comprise or represent one or more or a combination of computational methods that can be used to generate analytical models, classifiers and/or algorithms that lend themselves to solving complex problems such as, by way of example only but is not limited to, generating embeddings, prediction and analysis of complex processes and/or compounds; classification of input data in relation to one or more relationships. ML technique(s) may be additionally configured to enhance searches or used as part of a search algorithm or engine.

Typical search algorithm or engine may be accustomed to various data structures. These search algorithms or engine can be classified based on their mechanism of search dependent on the underlying data structures or heuristics. These algorithms may include but not limited to linear search, greedy (binary) search, digital search, and probabilistic searches such as Grover's algorithm. These search algorithms may be used in conjunction with or to supplement the various ML techniques herein described.

Examples of ML techniques that may be used by the invention as described herein may include or be based on, by way of example only but is not limited to, any ML technique or algorithm/method that can be trained on a labelled and/or unlabelled datasets to generate an embedding model, ML model or classifier associated with the labelled and/or unlabelled dataset, one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, reinforcement learning, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, Eclat algorithm, case-based reasoning, Gaussian process regression, gene expression programming, group method of data handling (GMDH), inductive logic programming, instance-based learning, lazy learning, learning automata, learning vector quantization, logistic model tree, minimum message length (decision trees, decision graphs, etc.), nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Naïve Bayes, hidden Markov models (HMMs), hierarchical hidden Markov model (HHMM), and any other ML technique or ML task capable of inferring a function or generating a model from labelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other an ML technique, task, or class of supervised ML technique capable of making use of unlabelled datasets and labelled datasets for training (e.g. typically the training dataset may include a small amount of labelled training data combined with a large amount of unlabelled data and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains and capable of learning or generating a model based on labelled and/or unlabelled datasets. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique capable of learning or generating a model based on learning data representations from labelled and/or unlabelled datasets.

The training of the ML models or classifiers may have the same or a similar output objective associated with input data. Data representative of the graph of entities/relationship is used as input labelled training datasets for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like.

For example, ML model(s) may be trained using one nor more ML techniques for expanding the search query associated with entities of interest and/or relationships thereto. The search query may include data representative of a first set of entities or entity concepts and the like. For example, an ML model may be configured to expand the search query by genericising and/or specificising the entities, entity concepts, terms of the search query and using these for expanding the search query. For example, the ML model may be generated from an ML technique by specific training data instances or labelled training data items from a training dataset for, by way of example only but not limited to, biological entities and/or relationships thereto. An example specific training data instance that may be used is based on, without limitation, for example a biological concept from a sentence (or text portion) of:
“Alzheimer's Disease is treated by modulating LRP1”
   In this example of a biological labelled training data item, the biological entity(ies) of interest in this portion of text include “Alzheimer's Disease” and “LRP1”. The relationship in this portion of text between these two entities of interest is described by “is treated by modulating”. Several biological relationship entities may be extracted and may include “is”, “treated”, “by”, and “modulating”. This training data item and a plurality of other training data items may be used to train an ML relationship extraction model for identifying and/or predicting further entities of interest and relationships thereto from a corpus of text or unstructured text (e.g. biomedical/biological documents, PubMed database(s), websites, articles etc.) for expanding the search query. This may output one or more sets of biological entity results including identified biological entities and relationships thereto and the like.

The biological entities of interest (e.g. “Alzheimer's Disease”, “LRP1”) may be genericised by selecting one or more entity(ies) associated with the biological entity of interest that are more generic and/or more specific than the biological entity of interest. However, it is to be appreciated by the skilled person that the biological entities of interest may also be specificised by selecting one or more entities associated with the biological entity of interest that are more specific than the biological entity of interest.

In this example, a hierarchical disease ontology based on the knowledge graph may be used to, by way of example only but not limited to, select several genericised entities associated with “Alzheimer's Disease”, where “Alzheimer's Disease”->“neurodegenerative disease”->“neurological disease”. The genericised entities associated with the biological entity of interest “Alzheimer's Disease” includes, by way of example only but are not limited to, “neurodegenerative disease” and “neurological disease”. These may be used to give one or more generalised text portions or sentences such as, by way of example only but is not limited to:
“neurodegenerative disease is treated by modulating LRP1”
“neurological disease is treated by modulating LRP1”
   Similarly, a gene ontology may be used to genericise the biological entity of interest “LRP1” for selecting several genericised entities associated with “LRP1”, where “LRP1” “lipoprotein”->“gene”. The genericised entities associated with the biological entity of interest “LRP1” includes, by way of example only but are not limited to, “lipoprotein” and “gene”. These may be used to give one or more generalised text portions or sentences such as, by way of example only but is not limited to:
“neurodegenerative disease is treated by modulating genes”
“neurological disease is treated by modulating lipoproteins”

Of course, various different combinations of the biological entities of interest and the selected genericised and/or specificised entities associated with the biological entities of interest may be used to generate different genericised sentences that could be used as labelled training data for training an ML model/classifier for learning generic patterns about diseases treated by modulating LRP1 (gene).

The above mentioned type of ML model(s) and/or technique(s) may be applied for generation of different genericised sentences, entities, entity concepts and the like for use in expanding the search query before generating knowledge graphs based on the expanded search query. Further ML model(s) and/or concepts may also be used to automatically generate or expand the search query. For example, ML model(s) using similarity and/or word vectors or word embedding (e.g. high dimensional, continuous space representation of word meaning) may be used and/or combined with one or more other ML model(s) (e.g. the above ML model) and/or systems and the like. In the case of word vectors or word embeddings, the word vectors/embeddings may be combined via a centroid that is the centre of the higher order representation (e.g. centroid of higher dimensional space representation) of all words together. For example, the centroid for “Heart disease>myocardial infarction>cardiac arrest” would be “heart disease”.

This can be taken further by genericising and/or specificising the biological relationship entities (e.g. sentence or non-biological entities), which in this example include, by way of example only but are not limited to, “is”, “treated”, “by”, and “modulating”. For example, alternative hierarchical data structure such as a grammar tree or syntax tree associated with the relationship “is treated by modulating” may be used to genericise each of the biological relationship entities. For example, each of the biological relationship entities may have genericised entities selected based on, by way of example only but is not limited to, “treated”->“verb”, “modulating”->“verb”, “is”->“conjunction” etc. This can lead to a multitude of further genericised sentences or portions of text based on the various combinations of all the biological entities and corresponding selected genericised entities associated with each biological entity. The combinations of the different portions of text may be used as labelled training data items for the above-mentioned specific training data instance/item. In addition, word embeddings may be generated for all of the biological entities (e.g. specific entities) and genericised entities associated with the biological entities in relation to the original text portion and combined to form one or more composite embeddings representing that text portion. This may be performed each time a text portion is required for input to a trained ML model or classifier, and/or for each training data item of a training dataset during training of an ML technique for generating an ML model or classifier.

Knowledge graphs generated may be used for training ML models for predicting, identifying and/or extracting one or more entities and relationships thereto from a corpus of text, and/or for training any other type of ML model configured for solving one or more classification problems, or objective problems and the like based on the knowledge graph as a training dataset. For example, by generating embeddings of both biological entity of interest and relationships information as graph forms (e.g. using information biological entities/relationship embedded within a graph.), means an ML model/classifier can leverage this information and learn how to interpret entity(ies) of interest and relationships thereto. Such embeddings allow ML models and/or classifiers to learn generic patterns in which certain patterns may have more relevance. For example, rather than the ML model being focused on a particular entity of interest (e.g. a disease such as “Alzheimer's Disease”), the ML model can robustly handle other related entity(ies) of interest (e.g. other neurodegenerative diseases) other than the particular entity(ies) of interest and relationships that it may have been trained on; the learnt patterns become transferable across a greater range of entity(ies) of interest (e.g. all neurodegenerative diseases or diseases and the like).

Although the embedding technique according to the invention is described herein in relation to biological entities such as, by way of example only but not limited to, entity(ies) of the entity type from the group of: gene; disease; compound/drug; protein; chemical, organ, biological; or any other entity type associated with bioinformatics or chem(o)informatics and the like, this is by way of example only and the invention is not so limited, it will be appreciated and understood by the skilled person that the invention is applicable to any corpus of text or literature, any type of one or more entity(ies) of interest within the text, relationships and/or subject-matter thereto, and/or as the application demands.

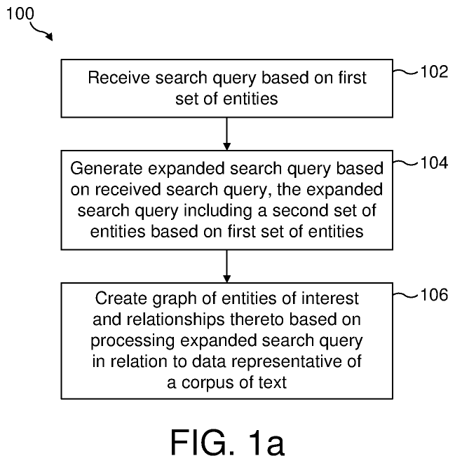

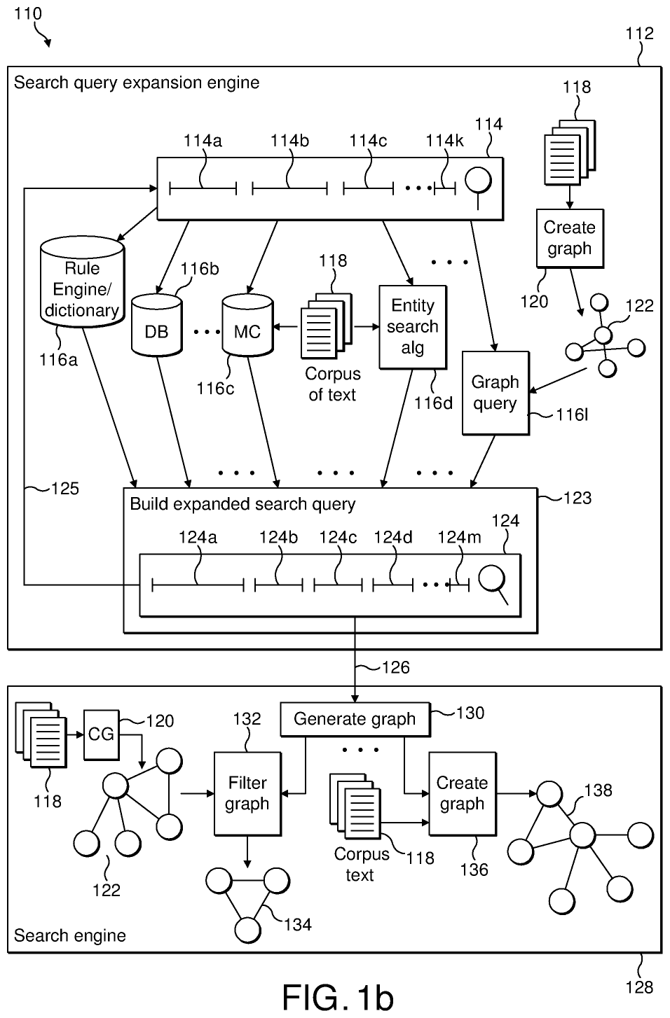

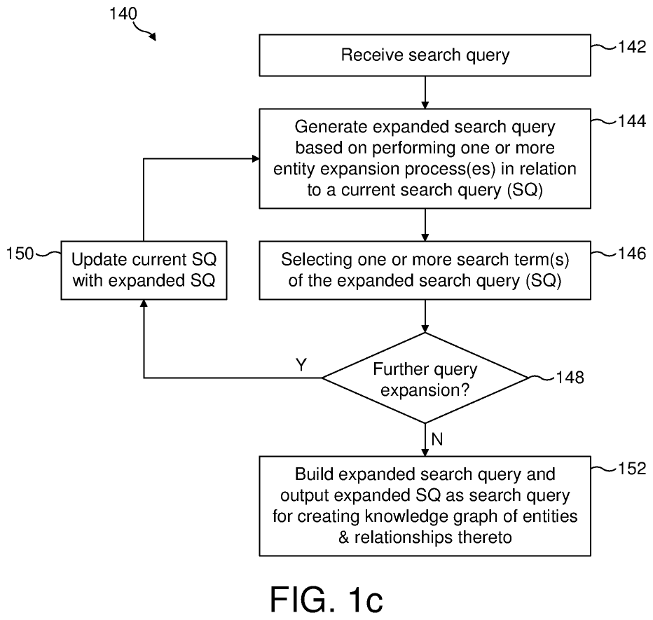

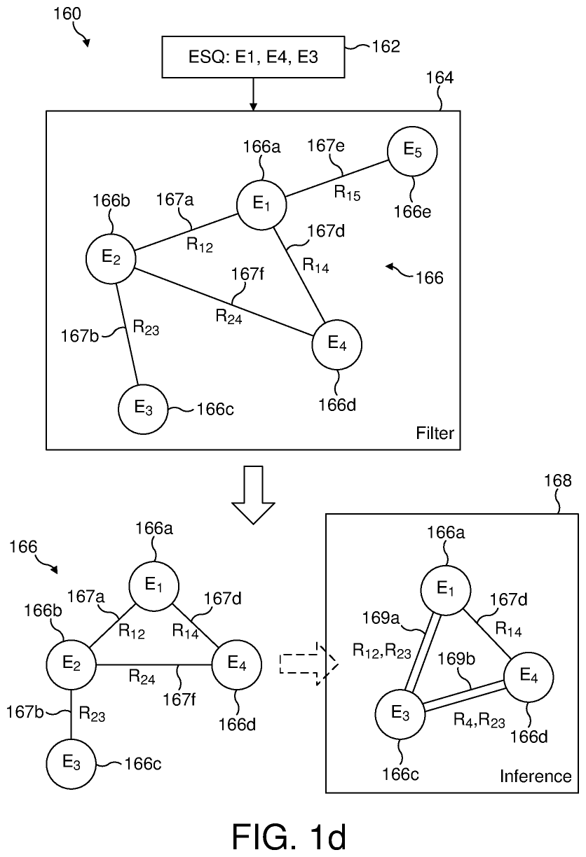

FIG. 1a is a flow diagram illustrating an exemplary process 100 for expanding a search query for creating a graph of entities of interest and relationships thereto from a corpus of text according to the invention. In step 102, one or more entity expansion process may receive a search query corresponding to entities of interest, where the search query comprising data representative of a first set of entities. In step 104, the process generates an expanded search query based on inputting the received search query to the one or more entity expansion process(es), where the expanded search query comprising data representative of a second set of entities and the first set of entities. In step 106, a graph of entities of interest and relationships thereto based on processing the expanded search query with data representative of a corpus of text or a portion thereof.

The graph of entities of interest and relationships may be created by retrieving a set of entities and relationships thereto from the corpus of text based on inputting data representative of the expanded search query to a search engine configured for identifying one or more entity(ies) and relationships thereto based on the received expanded search query and the corpus of text. In particular, this is accomplished by retrieving a set of entities and relationships thereto from the corpus of text. The input and output of the retrieval step are respectively the expanded search query to a document extraction engine configured for identifying portions of text from the corpus of text associated with the expanded search query, and one or more identified portions of text from the corpus of text associated with the expanded search query.

Alternatively or additionally, a set of entities and relationships thereto may be retrieved from the corpus of text using one or more ML extraction model(s) by way of generating predictions based on the expanded search query configured for predicting from the corpus of text a set of entity pairs and relationships associated with a set of entities associated with the search query. Each predicted entity pair comprising an entity of a first type and an entity of a second type having an associated relationship therebetween identified from the corpus of text. The predicted entity pairs and relationships are outputted as the set of entities and relationships. In one example, one or more ML, model(s) herein described may be used. In another example, the prediction may be based on one or more sets of rules. In yet another example, a hybrid system may include both ML model(s) and rule-based approaches. Effectively, this process provides (re)evaluation of the result set by way of robustly back-testing the predicted set of entities and relationships in order to improve accuracy of the prediction.

The identified portions of text from the corpus of text associated with the expanded search query to a relationship extraction engine may be configured for identifying or predicting one or more entity(ies) and relationship(s) thereto in relation to the identified portions of text associated with the expanded search query. The identified portions of text serve as input of the retrieval step whereas identified or predicted set of entity(ies) and relationship(s) may be outputted.

The corpus of text includes a plurality of entity types of interest in which each entity type has a corresponding set of entities that may be identified and/or extracted from the corpus of text. When these entities are identified/extracted from a portion of text, in cases from a corpus of text that may lack metadata and/or cannot readily be indexed or mapped onto standard database fields, and labelled to be a particular entity type of interest, then these entities may be used in many applications such as knowledge bases, literature searches, entity-entity knowledge graphs, relationship extraction, machine learning techniques and models, and other processes useful to researchers such as, by way of example only but is not limited to, researchers in the fields of bioinformatics, chem(o)informatics, drug discovery and optimisation and the like. The corpus of text may include, by way of example, but not limited to a collection of documents of natural language text. These documents may be may be partially structured. For example, a document may have structured headings for which together with portions of text from the document.

Portions of text may be a set of relevant documents from the corpus of text that are determined relevant to the entity concepts of the expanded search query. The relevant documents may be selected a number of ways. In one example, the search engine comprises one or more ML search model(s) is configured for identifying, predicting, ranking and/or scoring the plurality of documents associated with the expanded search query for determining the set of relevant documents. In another example, relationship extraction engine comprises one or more ML extraction model(s) configured for identifying, predicting, ranking and/or scoring a set of entities and relationships thereto in relation to the identified portions of the set of relevant documents and the expanded search query.

Alternatively or additionally, the relationship extraction engine may search through one or more existing database of relationships. Using the one or more existing database of relationships, a search may be performed to identify one or more entity(ies) and relationships thereto in relation to identified portions of the set of relevant documents and the expanded search query. Accordingly, the set of relevant documents may be determined based on the identified one or more relationships.

Furthermore, the search engine may comprise one or more information retrieval algorithms such as Term Frequency-Inverse Document Frequency (TF-IDF), that are associated with document frequency and/or document similarity for performing a document search. These information retrieval algorithms are associated with mining text and/or performing network analysis of digital libraries or databases. Varying weight scheme may be used in place of TF-IDF schemes such as Shannon entropy or entropy-based weighting term and the like.

An entity type may comprise or represent a label or name given to a set of entities that may be grouped together and share one or more characteristics, rules and/or properties and/or are considered to be listed under the same entity type. For example, in the bioinformatics and/or chem(o)informatics fields entity types may include at least one entity type from at least one of, by way of example only but is not limited to, a disease, gene, protein, compound, chemical, drug, biological pathway, biological process, anatomical region or entity, tissue, cell-line, or cell type, or any other biological or biomedical entity and the like; or any other entity type of interest associated with bioinformatics or chem(o)informatics entities and the like. In the data informatics fields and the like, an entity type may include, by way of example but not limited to, at least one entity type from the group of: news, entertainment, sports, games, family members, social networks and/or groups, emails, transport networks, the Internet, Wikipedia pages, documents in a library, published patents, databases of facts and/or information, and/or any other information or portions of information or facts that may be related to other information or portions of information or facts and the like.

An entity of interest may further comprise or represent an object, item, word or phrase, piece of text, or any portion of information or a fact that may be associated with a particular entity type and be associated with a relationship. An entity of interest may be, by way of example only but is not limited to, any portion of information or a fact that has a relationship, or a fact that has a relationship with another entity of interest, by way of example only but is not limited to, one or more portions of information or another one or more facts and the like. For example, in the biological, chem(o)informatics or bioinformatics space(s) an entity of interest may comprise or represent an entity based on an entity type such as, by way of example only but is not limited to, a disease, gene, protein, compound, chemical, drug, biological pathway, biological process, anatomical region or entity, tissue, cell-line, or cell type, or any other biological or biomedical entity and the like. For example, a biological entity of the biological entity type may be represented by data representative of a portion of text that describes or is descriptive of that biological entity type based on the context of the text portion or text in which that entity resides. A biological entity may include entity data associated with a biological entity type from one or more of the group of: gene; disease; compound/drug; protein; cells; chemical, organ, biological; or any other entity type associated with bioinformatics or chem(o)informatics and the like.

In one example, the first or second set of entities that pertains to the entities of interest may be associated with a set or corpus of text such as from patents, literature, citations or a set of clinical trials that are related to a disease or a class of diseases. In another example, in the data informatics fields and the like, the first or second set of entities may comprise or represent an entity associated with data informatics entity types such as, by way of example but not limited to, news, entertainment, sports, games, family members, social networks and/or groups, emails, transport networks, the Internet, Wikipedia pages, documents in a library, published patents, databases of facts and/or information, and/or any other information or portions of information or facts that may be related to other information or portions of information or facts and the like.

In another example, the first or second set of entities may be extracted from a corpus of structured text such as, by way of example but is not limited to, structured documents; database of patents or patent applications; web-pages; database of distributed sources such as the Internet; a database of facts and/or relationships; and/or expert knowledge base systems and the like; manually curated text or portions of text; and/or any other system or corpus storing and/or capable of retrieving portions of information or facts (e.g. entities of interest) that may be related to (e.g. relationships) other information or portions of information or facts (e.g. other entities of interest) and the like.

In a further example, entities of interest may be associated with the disease or gene entity type(s), in which the knowledge graph may be based on a disease or gene ontology in which a node at a certain level in the disease or gene ontology graph describes the entity of interest at a certain level of genericity or specificity, each parent node (or one or more ancestor node(s)) describing the entity of interest more generically, and each child node (or one or more descendant node(s)) describing the entity of interest more specifically. Example ontologies for specific biological entities may include, by way of example only but are not limited to, one or more gene ontologies for entity(ies) of the gene entity type such as, by way of example only but are not limited to, Gene Ontology (GO) from the Gene Ontology Consortium, GENIA ontology (e.g. xGENIA)—GENIA ontology may further include relationships between genes, and the like; one or more disease ontologies for entity(ies) of the disease entity type such as, by way of example only but are not limited to, The Disease Ontology (DO) from Northwestern University, Center for Genetic Medicine and the University of Maryland School of Medicine, Institute for Genome Sciences; one or more biological/biomedical entity ontologies or any other entity ontology based on, by way of example only but not limited to, the ontologies from the Open Biological and Biomedical Ontology (OBO) Foundry, which includes ontologies such as, by way of example only but not limited to, the Protein Ontology (https://www.ncbi.nlm.nih.gov/pmc/articles/PMC3013777/), or any type of ontology based on those from the Ontology Lookup Service (OLS) from European Molecular Biology Laboratory-European Bioinformatics Institute (EMBL-EBI), which includes ontologies associated with biological/biomedical entity types including, by way of example only but not limited to, gene, genomics, gene expression and the like; anatomical entities; disease, human disease and the like; antibiotic resistance; compound/drug; protein; cell; chemical; organ; food; biological; biomedical; or any other entity type associated with bioinformatics or chem(o)informatics and the like.

Expanded search query may be analyzed by syntax and/or via semantic associations. The expanded search query may comprise similar or closely related concepts and words derived from the seed term or the search query. The user may be permitted to provide substantive feedback of the query validity. The feedback may be incorporated in the iteration of further expansion. The expanded search query may be used either to extract or identify relevant documents, extract entities/relationships, and build a knowledge graph of entities of interest.

Graph of entities may be a graph with nodes as entities and edges as relationships. Such graph includes types, by way of example, that include but not limited to directed, undirected, vertex labelled, cyclic, edged labelled, weighted, and disconnected graph or subgraphs. Various algorithms may be used to traverse or search the graphs and determine the type of the graph or subgraph that is being generated. The type of graph generated may be learned using various ML techniques or models herein described.

Entity expansion process as illustrated in FIG. 1a permits a domain expert to generate new graphs or to update existing graphs (i.e. generate subgraphs of an existing graph) rapidly for a particular domain from related and relevant concepts and/or keywords through their initial search query or otherwise known herein as seed terms. The related and relevant concepts and/or keywords may be filtered using algorithms in conjunction with a text corpus to build the expanded search query of entities. The process or engine robustly suggest semantically similar concepts and words to expand the initial search query. Such entity expansion process may further use an existing graph of entities and/or sourced from other internal or external repositories as further illustrated in FIG. 1b. As such, process or engine improves the feasibility of generating adaptive entity-relationship graphs or knowledge graphs from unstructured data.

FIG. 1b is a schematic diagram illustrating an exemplary search system 110 for expanding a search query and creating a graph of entities 138 based on the process of FIG. 1a according to the invention. The data representative of the received search query may be sent to one or more entity expansion process 112. The entity expansion process may include, by way of example only but not limited to, one or more or a plurality of entity expansion process(es) 116a-1161 based on, without limitation, for example one or more rule-based engine/dictionary (lexicon) module 116a, internal or external repository 116b, an ML model 116c and/or graph entity search algorithm 116d/l that may use the corpus of text 118 to expand the search query. In particular, the search query 1161 may perform the expansion process based on an existing graph of entities 122, where the existing graph 122 of entities of interest and relationships thereto is previously generated based on the corpus of text 118. The output entities, entity concepts, words, terms or phrases of the entity expansion process(es) 116a-1161 may be used by the build expanded search query module 123 to form a second set of entities 124 including a plurality of entities 124a-124m that form an expanded search query. The build expanded search query module 123 may be configured to validate the output entities, entity concepts, words, terms or phrases of the expansion process(es) 116a-116l when building the second set of entities 124 of the expanded search query. Additionally, the second set of entities 124 of the expanded search query may be fed back 125 for validation and/or further search query expansion may be performed again where those validated entities, concepts and terms for the search query of the second set of entities 124, first set of entities 114, are used or are merged or used inconjunction with each other input to the entity expansion process(es) 116a-1161 for generating further sets of entities to further expand the search query. The search query expansion may be iterated multiple times using feedback 125 for iteratively generating the expanded search query 124. The expanded search query 124 in each iteration corresponds to entities of interest based on a selection of data representative of the second set of entities 124a-124m and the first set of entities 114 in relation to the entities of interest. These may be validated by the build expanded search query module 123. The feedback 125 from the expanded search comprises validated entities and concepts associated with the knowledge graph that provides enhanced recall and improved accuracy by expanding the search space while maintaining the same or better level of precision.

For example, during a first iteration of the entity expansion process(es) 116a-1161, a current search query 114 is received by the system 110. Data representative of the second set of entities 124a-124m based on the current search query 114 is received from the one or more entity expansion process(es) 116a-1161. An expanded search query corresponding to entities of interest based on a selection of data representative of the second set of entities 124 and the first set of entities 114 in relation to the entities of interest is built and/or validated by the build search query module 123 and the current search query 114 is updated as the iteration continues. Once the search query 114 has been sufficiently expanded (e.g. there is no more improvement in the number of terms or quality of terms or relevance of term found by the expansion process(es) 116a-1161, and/or a user indicates the expanded search query is suitable) then the expanded search query 124 is output and feed to the search engine 128, which performs a search based on the expanded search query 124 for building one or more search results in the form of one or more knowledge graphs and/or sub graphs 134, 138 and the like. These are output from the search engine 128 in response to the initial search query.

Further, in FIG. 1b, once search query expansion is completed, the search engine 128 receives the expanded search query 124 and performs a search based on the expanded search query 124 to output one or more knowledge graph 138 or sub graphs 134 that may be built or generated 120/130/136 from expanded search query 124. This may be performed using generate graph module 130, which is configured to use search graph index based on existing graphs of entities and/or create additional graphs of entities that may be used for processing the expanded search query 124. For example, a create graph module 120 may generate or update knowledge graph 122 based on a corpus of text 118 in relation to multiple entities, entity types of interest and the like. The graph 122 may be periodically or continuously updated as the corpus of text 118 changes. The graph 122 may form a search graph index or database from which the expanded search query 124 may be processed with. For example, the filter graph module 132 may use the graph 122 and the expanded search query 124 to generate a filtered graph 134. The filtered graph 134 may be output as the search results in relation to the expanded search query 124. Alternatively or additionally, a create graph module 136 may be configured to process a corpus of text 118 based on the expanded search query 124 to generate graph of entities of interest 138. This may be output as the search results in relation to the expanded search query 124. Additionally or alternatively, graphs 134 and/or 138 may be used to update and/or build upon existing knowledge graphs 122 or for creating new knowledge graphs (not shown) and the like.

In various examples, the knowledge graph 138 or subgraphs 134 may be generated based on existing graphs of entities 122, which are filtered using the expanded search query 124. In both cases, the underlying graph representation of the entities/relationship may continuously update the knowledge graph 138 or subgraphs 134 from various technical fields that include but not limited to biology, biochemistry, chemistry, medicine. Knowledge pertaining to the corpus of text 118 may be updated and presented graphically as knowledge graph 138 or sub graphs 134 retaining the entities/relationships extracted from the corpus of text 118. In effect, one or more entity expansion process systematically and iteratively add representative entities to the expanded search query 124 while minimizing undesired redundancy. For example, it is not necessarily known before transitioning to a vertex of a knowledge graph that it has already been explored. As a graph become denser through updating, this redundancy becomes more prevalent, causing computation time to increase. Therefore, filtering an existing graph of entities of interest and relationships effectively cut down this time required. For example, the filtering may additionally or alternatively apply graph traversal with the heuristic similarity being based on, without limitation, for example semantic similarity (e.g. cosine similarity) of two specific terms, nodes or entities of the nodes. For example, one node may be more similar to another based on, without limitation, for example the cosine similarity of the two continuous representations and the like. Although cosine similarity is described herein, this is by way of example only but the invention is not so limited, it is to be appreciated by the skilled person the any other suitable type of heuristic and/or semantic similarity may be used or applied as the application demands.

The entity expansion processes 116a-1161 are configured to suggest semantically similar concepts and words, via one or more above-described entity expansion process, to expand the initial search query or seed terms based on a set of criteria that is dependent on the relative similarity and relevance of the word pairs. Relative similarity may be derived from one or more similarity metrics. On the other hand, this set of criteria is assessed based on a statistical distribution, i.e. Gaussian distribution, in accordance with a metric associated with the set of criteria. In essence, without limitation, for example expansion of the search query may use one or more similarity metrics. As the expansion proceeds, the increased volume of text from the corpus of text can improve the accuracy of the search expansion (and/or one or more similarity metrics) by providing more context to the underlying words, terms, entities and/or relationships and the like. Additionally or alternatively, other parameters may be used such as, without limitation, for example the amount of sub-word information i.e. the characters that create the concepts and/or words (a superset of morphemes) may be used to learn from, assess and/or examine combinations of concepts/words and the like. For instance, if a word does not appear in the corpus of text, it could infer the meaning of the neologism by identifying prefixes and suffixes that may pertain to a sub-word.

In operation, a search query comprising the seed terms may be received by the graph query. The seed terms are expanded based on terms inherent to an existing graph of entities, preferably trained on a corpus of text either structured or otherwise. The graph query similarly expands or build the expanded search query in conjunction or in combination with the above-mentioned one or more entity expansion process. In addition, the expanded search query may be fed back to the user as the user can either add or reduce the expanded search query and expansion process is iterated. From the expanded search query, a search is performed for entities of interest and relationships thereto in the corpus of text based on the expanded search query. This, in effect, forms or generates the graph of entities of interest and relationships thereto based on search results output from said search. The graph of entities of interest and relationships may be filtered based on the expanded search query, where the existing graph of entities of interest and relationships thereto is previously generated based on the corpus of text.

In one example, the entity expansion process may expand the seed term to incorporate and supplement from a database or lookup table of associations with biological concepts. In another example, an algorithm that scrape (search and extract) from the text corpus or ML model that learn from the text corpus may be used to predict additional biological concepts. In a further example, expansion may be derived from an algorithm that generates a knowledge graph or produces a sub graph from a corpus of text. Alternatively, the expansion process may be a combination any two or more of the above exemplary methods, however does not limit to only these methods. In addition, user may select from the predicted or expanded set of biological concepts as feedback to the entity expansion process deduce a more accurate set of expanded search query.

FIG. 1c is a flow diagram illustrating an exemplary process 140 for search query expansion based on the process and search system of FIGS. 1a and 1 b according to the invention. In step 142, the process and search system receives a search query. The process and search system, in step 144, generate expanded search query based on performing one or more entity expansion process in relation to the current search query obtained from step 142. By selecting one or more search term(s) of the expanded search query in step 146, process and search system determine, in step 148, if further query expansion is required or expanded search query receives feedback that one or more of the entities of interest of the expanded search query are valid. If so, in step 150, the process and search update the expanded search query to only include data representative of the valid entities of interest. Alternatively, if no further query expansion is required, then build the expanded search query and output expanded search query in step 152. In this step, built search query may be used to generate the graphs of entity and relationship based on the text corpus.

As the expansion of the search query may be performed iteratively through multiple steps via the one or more entity expansion processes, such feedback/update as illustrated in FIG. 1c may be essential as to disregard dissimilar or not so related entities to be included within final set of entity concepts. The selection of one or more search terms may be of a distribution. For example, the distribution may be binary corresponding to either valid or not valid. Alternatively, other distribution may be used for the purpose of selecting one or more search terms of the expanded search query.

FIG. 1d is a schematic diagram illustrating an example of creating a graph 166 based on filtering an existing graph of entities of interest 164 and relationships thereto in relation to the expanded search query 162 of FIGS. 1a to 1c according to the invention. Here, based on the expanded search query 162 (e.g. entities or entity concepts E1, E4, E3) search results may be derived from performing a search for entities of interest and relationships, relevant entities pairs and their relationships that may be extracted from graph 164. Graph 164 may be generated by extracting from the text corpus a plurality of entities of interest and relationships, relevant entities pairs and their relationships and embedded onto a graph 164. This graph 164 of entities of interest and relationships are formed showing, by way of example only but not limited to, a series of nodes (entities E1 to E5) and edges (relationships R12 to R24). Following the formation of the graph 164, the graph 164 may be filtered based on the expanded search query 162. For example, the edge nodes (i.e. node for E5 166e) may be disregarded by the filter, and alternatively, inferences 168 could be made with regards to edge nodes (i.e. between node for E3 166c and node for E4 166d) based on the existing relationships (i.e. R12, R14, R24, R23). The resulting sub-graph 168 may then be output as the search results in response to the expanded search query 162. The graph 164 may be continuously updated with regards to the search results based on the expanded search query 162 and/or from extracting entities and relationships thereto from the text corpus 118 based on the expanded search query 162 or other extraction process(es). This permits domain experts to effectively update or generate sub-graphs without having to recreate the entire graph 162. In another example (not shown in the figure), concepts, words or entity concepts/entities such as drugs may be filtered out based on a similarity metric and the like. This may assist in providing the system with more information on what the concept is. The filter may be based on, without limitation, for example semantic similarity (e.g. cosine similarity) of these concepts, words, and phrases in accordance to the one or more similarity metrics as described. For example, using semantic similarity (e.g. cosine similarity), the similarity between the concepts, for example, between the drug “Tylenol” and a disease may be determined and the like. Although cosine similarity is described herein, this is by way of example only but the invention is not so limited, it is to be appreciated by the skilled person the any other suitable type of heuristic and/or semantic similarity and the like may be used or applied as the application demands.

Performing a search for entities of interest and relationships traversing a graph, e.g. traversing graph 164, may be accomplished by adaptations of breadth-wise or depth-wise algorithms that are typically for searching a tree data structure. In either case, starting from a node, the algorithm visits every other node and returns to the starting node. For example, breadth-wise search or typically as breadth-first search starts a node of the graph, the search explores all of the neighboring nodes at the present depth prior to moving on to the nodes at the next depth level. Alternatively, a depth-wise search may be performed or in such cases applying a combination of both depth and breadth. In addition, above-noted ML techniques may be applied during the performance of the search for entities of interest and relationships to reduce the number of computation required during the search process.

FIG. 1e is a schematic diagram illustrating another example of a system 170 for creating a graph of entities of interest and relationships 176 thereto in relation to the expanded search query 162 of FIGS. 1a to 1c according to the invention. A corpus of text 172 is used in conjunction with the expanded search query 162 for generating entity results 174b comprising one or more entities and relationships thereto. As illustrated, extraction module 174 receives the expended search query 162 and text portions from a corpus of text 172 in which an identification and/or extraction module 174a performs extraction and/or identification of entities and their relationships using various techniques, such as ML model(s), rule-based system(s), existing knowledge graphs and the like. Entity results 174b that are derived from the entity extraction module 174a using the corpus of text 172 and the search results 162 are used to create the graph 174 of entities of interest and relationships thereto. The entity results 174b may be either stored as data representative of entities and relationships thereto. In this example, the entity results 174b may form a set of entities and relationships thereto. For example, the set of entities includes, without limitation, for example: a first pair of entities E1 and E5 and entity relationship R15 therebetween; a second pair of entities E2 and E3 and entity relationship R13 therebetween; a third pair of entities E1 and E2 and entity relationship R12 therebetween; a fourth pair of entities E9 and E1 and entity relationship R14 therebetween; and so on, to an N-th pair of entities EN and Ei and entity relationship RNi therebetween. This list may include one entity with a relationship thereto, that links to itself. Additionally or alternatively, the entity results 174b may be processed and/or passed along to form a graph of entities of interest and relationships thereto 176. In particular, the set or list of relationships and entity pairs such as E1 to E5, Ei and EN are extracted 174a with their corresponding relationships R12 to RNi. Based on the entity pairs and the corresponding relationships, the graph 176 is formed from the entity results 174b. The graph 176 includes a plurality of entity nodes 176a-176e and relationship edges 177a-177f, each entity node is linked to another entity node by a relationship edge. In this case, graph 176 includes two disconnected/undirected graphs of entities of interest and relationships thereto are presented with nodes 176a-176g and edges 177a-177f based on entities E1 to E5 and Ei and corresponding relationships R12, R15, R23, R14 to RNi therebetween.

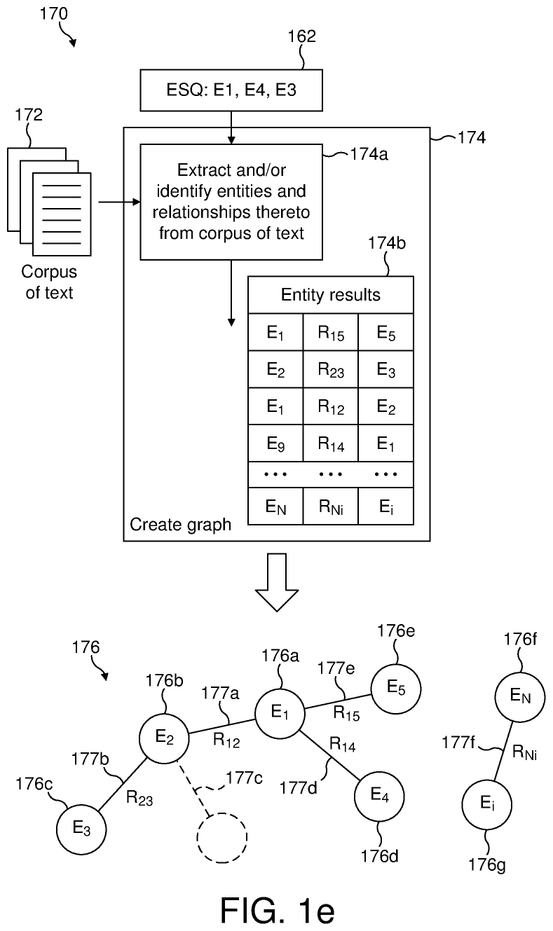

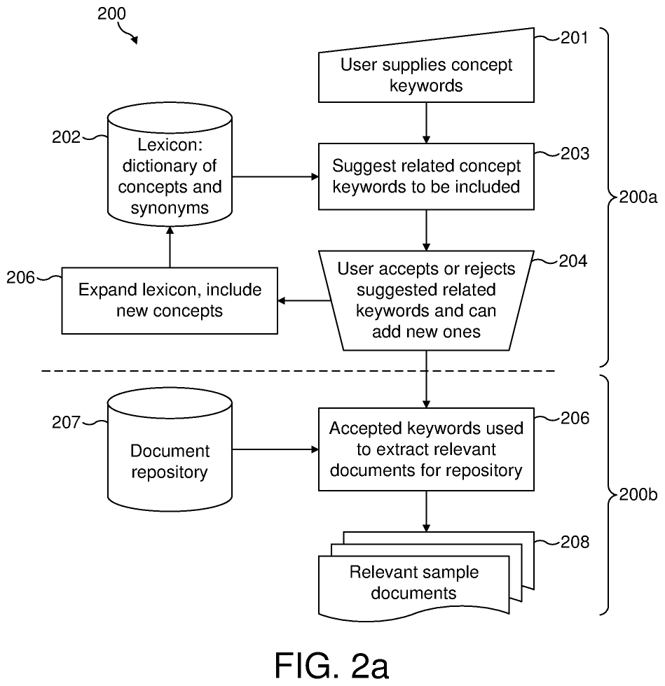

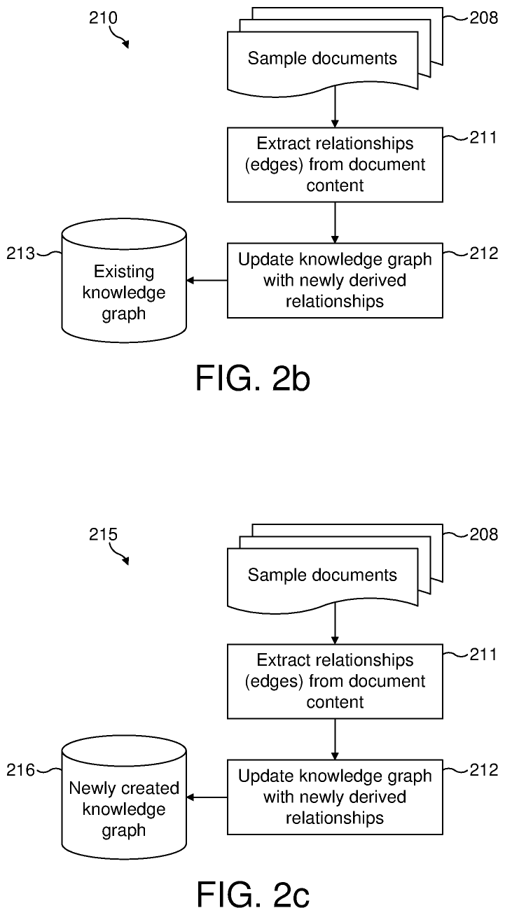

FIG. 2a is a schematic diagram illustrating another example search system 200 for automatically expanding key terms of biological concepts of a search query and retrieving relevant documents from a document repository based on the search query according to the invention. The search system 200 includes lexicon expansion 202a, document relevancy search 202b, and knowledge graph generation 210 or 215 in FIGS. 2b and 2c. Referring to FIG. 2a, in this example, lexicon expansion 202a includes the user providing the initial seed terms or key words 201 associated with entities or entities of interest. A lexicon system 202 suggests additional keywords synonymous to induce feedback from the user and provides or displays 203 these to the user for feedback. The feedback may either accept or reject 204 the suggested key words as valid, and/or include new key words and the like. The lexicon may be expanded and updated 205 and 204 to include the new accepted concepts or keywords from the user in relation to the original set of keywords. This may involve updating one or more dictionaries of concepts and synonyms, and/or rules associated with the lexicon system 202 and the entities/keywords accepted and/or rejected. The lexicon system 202 is updated continuously based on the validity of concepts or keywords. For example, if a user rejects a concept as not valid, the concept may be deemed unrelated to the concept originally presented as an input, the lexicon system 202 may be updated to dissociate the two concepts from each other. This process is iterative as the list of key words are continuously updated.

Once a list of keywords has been finalised or at any point during the iterative process where the list of key words are deemed sufficient. The list of keywords associated with one or more entities of interest may be used to perform a document relevancy search 200b, in which a corpus of text 207 or document repository is searched based on the list of accepted keywords. The document relevancy search 200b may be based on ML document extraction/search model(s) and/or rule-based document search system(s) for extracting a set of relevant documents or portions of text from the corpus of text 207 based on the accepted keywords and the like. The output of the document relevancy extraction 200b may be a final sample set of relevant documents that are considered the most relevant documents in relation to the set of keywords, which may then be used to extract relationships between concepts such as, for example, one or more entities and relationships thereto associated with the keywords and the like. The final sample set of relevant documents may be based on ranking a plurality of documents output from the ML document extraction model(s) and/or rule-based system(s), where the topmost ranked documents of the plurality of documents form the final sample set of relevant documents.

FIGS. 2b and 2c are schematic diagrams illustrating relationship extraction system 211 and knowledge graph generation systems 212 for generating/updating a knowledge graph associated with entities and relationships thereto from the final sample set of relevant documents 208. The relationship extraction system 211 is configured for extracting (e.g. biological) entities and associated relationships from the final set of relevant documents 208 retrieved from document relevancy search 200b of FIG. 2a according to the invention. The entities and associated relationships may be extracted as a set of entities and/or relationships thereto, which are processed by knowledge graph system 212 for generating and/or updating a knowledge graph with newly derived entity relationships and/or entities with relationships to other entities within the knowledge graph and the like. FIG. 2b shows update of an existing knowledge graph. While in FIG. 2b, the existing graph is updated 213, FIG. 2c illustrates new graph 216 may be created. Effectively, using the expanded search query, edges (relationships) between pairs of entities of interest are extracted from the final set of sample documents extracted from the text corpus 207. These are used to update and/or create a knowledge graph 213 and/or 216, respectively.

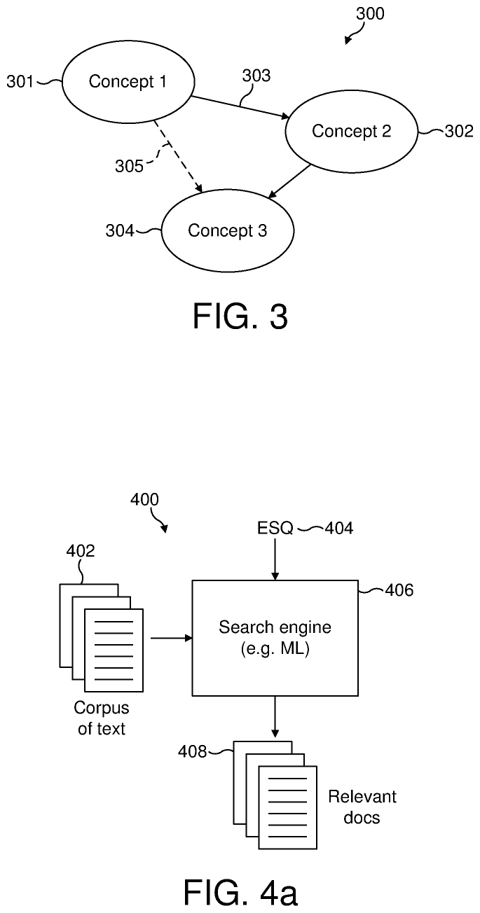

FIG. 3 is a schematic diagram illustrating an example knowledge graph 300 associated with concepts and corresponding relationships thereto according to the invention. Here, the knowledge graph comprises three nodes 301, 302, and 304. Respectively, the nodes are based on a set of entities that are shown as concepts 1, 2, and 3 in the figure. Solid edges of graph 303 represent extracted relationships between nodes correspond to a particular relationship between the entities represented by the pair of concepts.

Further in FIG. 3 is a dashed edge 305 that illustrates an inferred relationship from the existing nodes and relationships or through other above-noted means. In particular, the graph may infer a relationship edge between concept 1 of the first node 301 and concept 3 of a second node 304 of the graph when a first relationship edge exists from the first node to another node of the graph, and a second relationship edge exists from another node to the second node. An inferred relationship edge is inserted between the node pairs as a dashed edge 305.

The inferred relationship edge may be inferred, for each node of the plurality of nodes in the graph, between each node and another node of the graph when a relationship edge path exists from said each node via one or more further nodes to the other node. The inference may be derived probabilistically or through any other method/techniques/algorithms as described above. The inferred relationships are not node dependent (e.g. not necessarily only requiring a direct relationship/single edge therebetween), which means the concept itself may be updated and any node semantically below the concept will also be updated. The inferred relationships may traverse more than one node of the graph (e.g. traverse a path from a start node, via one or more nodes, to an end node in the graph). The graph may be updated based on the inferred relationship, where the inferred relationship edge is inserted between said each node and the other node of the graph (e.g. between a start node and the end node of the graph).

In particular, the relationship edge between each pair of nodes may be weighted. By weighting each relationship edge between each pair of nodes of the graph based on detecting the number of common relationships between the entities of said each pair of nodes from the set of entities and relationships, the inferred relationship edge may be more accurately assessed.

In one example, the knowledge graph may be presented graphically to the user. Alternatively or additionally, the knowledge graph results or data may be stored in the structured database for assessing using, for example, query languages. In either example, validated entities or concepts associated with the knowledge graph may be fed back into the search query expansion process to provide enhanced recall and improved accuracy. This is done by increasing the coverage without increasing ambiguity of the search. For instance, the validated entity may improve accuracy by reducing case where an acronym for a drug may be the same as the acronym for another entity.

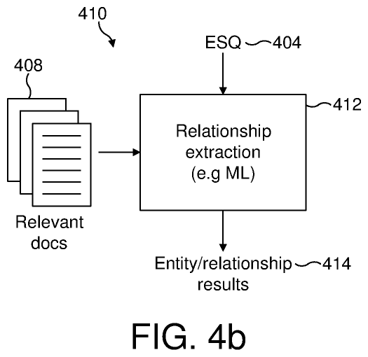

FIG. 4a is a schematic diagram illustrating an exemplary document relevancy engine 400 (e.g. ML search model) for use with FIG. 1a-3 according to the invention. Not shown in the figure is a graph of entities of interest and relationships thereto comprising a graph structure comprising a plurality of nodes based on a set of entities, where each node of the graph structure represents an entity and edges between a pair of nodes correspond to a particular relationship between the entities represented by the pair of nodes. As illustrated in FIG. 4a an expanded search query 404 may be input to a document relevancy search model 406 configured for extracting and/or identifying documents associated with an expanded search query from a corpus of text 402. Using the expanded search query 404 the document relevancy search model 406 may conduct searches and retrieve a set of relevant documents that include entities and relationships thereto associated with the expanded search query from the corpus of text (402). ML model 404 is configured to predict, extract and/or identify additional relevant documents 408 from the corpus of text 402 and the like.

FIG. 4b is another schematic diagram illustrating an exemplary relationship extraction system 410 (e.g. ML relationship extraction model 412) for use with FIG. 1a-3 and in conjunction with FIG. 4a according to the invention. Following FIG. 4a, the relationship extraction system 410 generates entities/relationship results 414 from the relevant documents 408 together with the expanded search query 404 using techniques such as ML relationship extraction model(s) and/or named entity recognition model(s). The ML relationship extraction model(s) is configured to predict or identify entities of interest and relationships thereto based on the expanded search query and the relevant documents 408. Similarly, ML based named entity recognition system(s)/model(s) may be used to identify and/or extract entities from the relevant documents 408 and relationships thereto.

In one example, rather than having two separate ML model(s) and/or systems 400 and/or 410 for identifying relevant documents 408, then results of entities and relationships thereto 408 from the relevant documents 408 using the above-described ML models of FIGS. 4a and/or 4b, these ML model(s) may be replaced by an ML model configured for generating a set of entities and relationships thereto based on the expanded search query and a corpus of text 40. For example, the ML model may be configured for predicting and/or identifying from the corpus of text a set of entity pairs and relationships associated with a set of entities associated with the search query, each predicted/identified entity pair comprising an entity of a first type and an entity of a second type having an associated relationship between identified from the corpus of text 402. The set of entity pairs and relationships as the set of entities and relationships are generated and outputted. The set of entity pair and relationships may be used for, without limitation, for example updating and/or building knowledge graphs 213 and/or 216 of FIGS. 2c and 2b and the like.

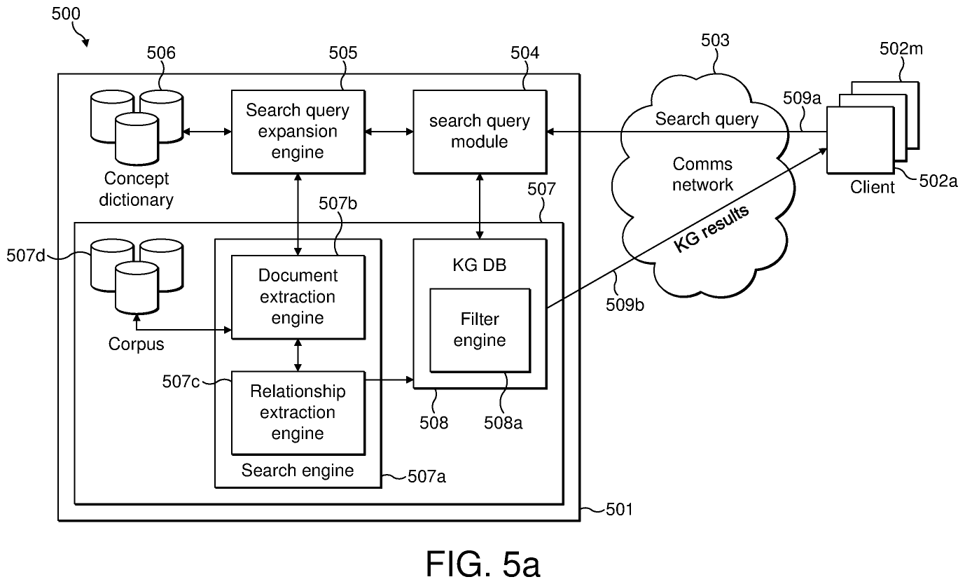

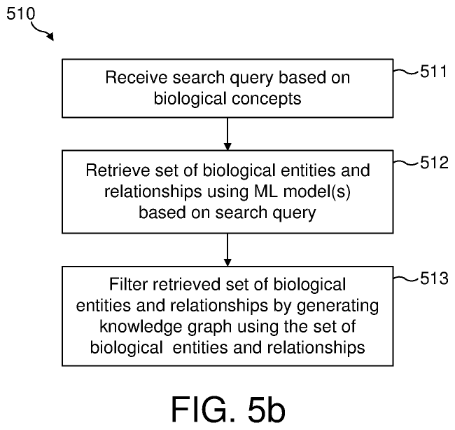

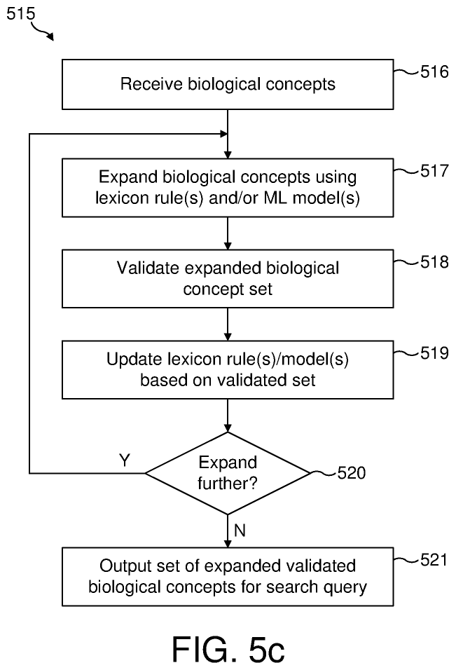

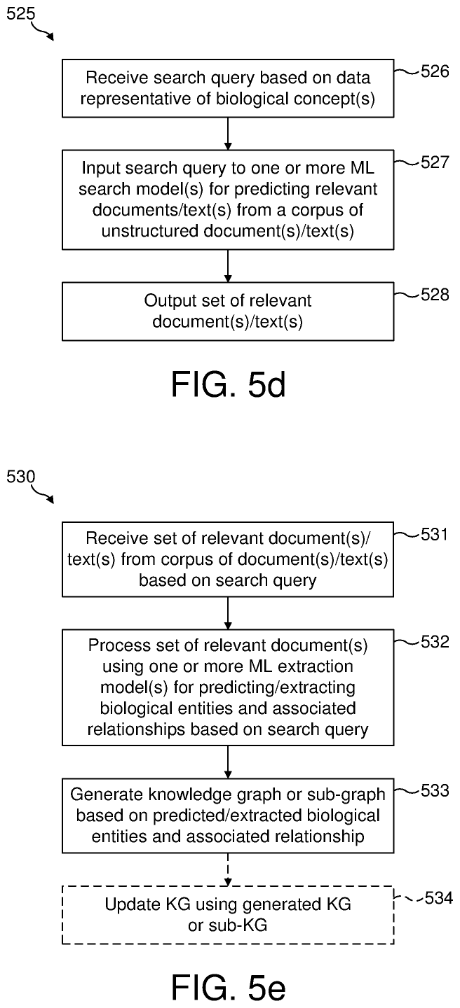

FIG. 5a is a schematic diagram illustrating a further example search system 500 according to the invention. The system 500 comprises a plurality of client device(s) 502a-502n in communication over a communication network 503 with a knowledge graph search system 501. The knowledge graph search system 501 includes a receiver component 504 that is configured to receive a search query 509a from a user of a client device 502a corresponding to keywords associated with entities of interest and/or relationships thereto and the like. For example, the search query may include data representative of a first set of entities. One or more search queries may be sent from the client devices 502a-502n module via a communication interface through a network 503. Each search query 509a may be received via the search receiver component 504, which is configured to either determine whether search query expansion 404 should occur, and/or whether the search query 509a may be processed using an existing knowledge graph search index or database 508 of graph search index creation/update component 507. In particular, the search query expansion component 505 is configured to generate an expanded search query based on inputting the received search query 509a to one or more entity expansion process(es), the expanded search query comprising data representative of a second set of entities and the first set of entities. For example, the search query expansion component 505 may be configured to include, without limitation, for example the search expansion step 104 of FIG. 1a, search query expansion engine 112 of FIG. 1b, process 140 of FIG. 1c, and/or lexicon expansion system 200a as described with reference to FIGS. 2a to 4b.

In particular, the one or more entity expansion process(es) includes but not limited one or more rule based engine, internal or external repositories, ML model(s), corpus of structured or unstructured text, entity search algorithm(s), and knowledge graph based expansion process as described in FIG. 1b for search query expansion engine 112 and/or as described with reference to FIGS. 1a to 4b. For example, as shown in FIG. 5a, the one or more entity expansion process(es) as described herein may use a concept and/or entity dictionary 506 and/or be a lexicon system that uses one or more concept and/or entity dictionaries 506 for suggesting search concepts, terms and/or entities in relation to expanding the search query 509a.

Further in FIG. 5a, graph search index creation/update component 507 is configured to create a search index graph of entities of interest and relationships thereto based and/or updating a search index graph of entities of interest and relationships thereto based on processing the expanded search query associated with search query 509a that is output from the search query expansion component 505. For example, the graph search index creation/update component 507 may be configured to include, without limitation, for example the graph creation/update step 106 of FIG. 1a, graph search engine component 128 of FIG. 1b, graph process(es) 140 or 170 of FIGS. 1c or 1 d, and/or document relevancy search 200b and/or graph creation/update systems 210 and 215 as described with reference to FIGS. 2a to 4b.

In this example, the graph search index creation/update component 507 may include, by way of example only but is not limited to, a search engine 507a and a filter engine 508a. The search engine 507a includes a document extraction engine 507b and relationship extraction engine 507c. The search engine 507a includes the document extraction engine 507b that receives input from a corpus of text 507d. In particular, the document extraction engine 507a processes the expanded search query associated with the search query 509a and the corpus of text 507d to generate a set of relevant documents in relation to the search query 509a. The set of relevant documents being the most relevant documents associated with the search query 509a based on the expanded search query therewith. For example, the document extraction engine 507b may be configured to include, without limitation, for example the functionality as described in relation to steps or portions of the graph creation/update step 106 of FIG. 1a and/or graph search engine component 128 of FIG. 1b, and/or document relevancy search 200b as described with reference to FIG. 2a and/or corresponding models and/or systems as described with reference to FIGS. 3 to 4b. The set of relevant documents is consequently processed by the relationship extraction engine 507c to derive entities/relationships from the set of relevant documents. For example, the relationship extraction engine 507c may be configured to include, without limitation, for example the functionality as described in relation to the steps or portions of the graph creation/update step 106 of FIG. 1a and/or graph search engine component 128 of FIG. 1b, process 170 of FIG. 1d, and/or relationship extraction 211 of graph creation/update 210 and/or 215 as described with reference to FIG. 2b and/or 2c and/or corresponding models and/or systems as described with reference to FIGS. 3 to 4b.

The generated entities/relationships are further processed through the filtering engine 508a to generate and/or update a search index knowledge graph. Knowledge graph search index database 508 is configured to process the expanded search query of the search query 509a and produce graph results 509b that are fed back to the client devices 502a-m to which initially input the search query 509a via the network 503. The results that are fed back are validated so as to improve accuracy and enhance recall. The entire process may be iterative as to expand search queries and to update the knowledge graph search index.

FIG. 5b is a flow diagram illustrating an exemplary process 510 for searching and filtering biological entities of interest from a corpus of text for use with the search systems of FIGS. 1a-5a according to the invention. In step 511, the search system receives search query based on biological concepts. In step 512, based on the search query, the ML models effectively retrieve a set of biological entities and relationship. In step 513, the retrieved set of biological entities and relationships are filtered and knowledge graph are generated using the biological entities and relationships.

FIG. 5c is a flow diagram illustrating another example process 515 for expanding biological concept search query of FIG. 5a according to the invention. In step 516, the search query expansion engine receives the biological concepts. In step 517, the engine expands the biological concept using lexicon, rules(s) and/or ML model(s). In step 518, the engine validates the expanded biological concept set. In turn, lexicon, rules(s) and/or ML model(s) are updated based on the validated set in step 519. In step 520, the steps 517 to 519 are iterated until the expansion of the concept is no longer required or meets certain criteria of validation. The set of expanded and validated biological concepts is ready for the search engine to extract the entities/relationships and generation of the knowledge graph as output 521.

In one example, a current set of biological concepts or entity concepts is expanded based on the expansion engine configured to expand the current set of biological concepts into data representative of a further relevant set of biological concepts, where in the first iteration the current set of biological concepts is the first set of biological concepts. The biological concepts or the entities representative thereof includes, by way of example only but are not limited to: gene; disease; compound/drug; protein; chemical; organ; biology; biological part; or any other entity type associated with bioinformatics, chem(o)informatics, biology, biochemistry, chemistry, medicine, pharmacology, and/or any other field relevant to diagnostic, treatment, and/or drug discovery and the like. The expansion engine receives feedback that one or more of the biological concepts from the current set of biological concepts and/or further relevant set of entity concepts are valid or of interest as described. The expansion engine generates an expanded set of biological concepts based on the validated or of interest entity concepts from the current set of entity concepts and/or further relevant set of entity concepts. The expansion engine replaces the current set of entity concepts with the expanded set of entity concepts. Iteratively performing the steps of expanding the current set of biological concepts, receiving feedback, and generating an expanded set of biological concepts until a stopping criterion in relation to expanding the current set of entity concepts is reached. Finally, the expansion engine generates an expanded search query based on the current set of biological concepts.

FIG. 5d is a flow diagram illustrating an example process 525 for searching for relevant documents from the corpus of text based on the search system and/or search query of FIGS. 5a-5c according to the invention. In step 526, the expanded search query is received and based on data representative of the biological concepts. In step 527, the expanded search query is inputted to one or more ML search Model(s) for predicting relevant documents/texts from a corpus of document/texts. The predicted relevant documents/texts are output for the purpose of extracting entities/relationships associated 528.

In one example, biological concepts are derived from the portions of text may include a set of relevant documents from the corpus of text that are determined relevant to the entity concepts of the expanded search query. The relevant documents may describe concepts that include but not limited to gene; disease; compound/drug; protein; chemical; organ; biology; biological part; or associated with bioinformatics, chem(o)informatics, biology, biochemistry, chemistry, medicine, pharmacology, and/or any other field relevant to diagnostic, treatment, and/or drug discovery and the like. Accordingly, one or more ML search model(s) may be configured for identifying, predicting, ranking and/or scoring the plurality of documents associated with the expanded search query for determining the set of relevant documents.

FIG. 5e is a flow diagram illustrating an example process 530 for processing the relevant documents of FIG. 5d for extracting biological entities and associated relationships for creating a graph of entities of interest and relationships thereto according to the invention. In step 531, relationship extraction engine receives a set of relevant documents/text from corpus of documents/texts based on the search query. In step 532, the relationship extraction engine processes set of relevant documents using one or more ML extraction models for predicting/extracting biological entities and associated relationship based on the search query. In step 533, knowledge graph and/or subgraphs are generated based on predicted/extracted biological entities and associated relationships. In step 534, optionally, the knowledge graph is updated via sub-graphs or new knowledge graph.

In one example, relationship extraction may include receiving one or more identified portions of text from the corpus of text associated with the expanded search query to a relationship extraction engine configured for identifying or predicting one or more biological entities and their relationships thereto in relation to the identified portions of text associated with the expanded search query. With the above ML extraction models configured for identifying, predicting, ranking and/or scoring a set of entities and relationships thereto in relation to the identified portions of the set of relevant documents and the expanded search query, relationship extraction engine outputs the identified or predicted set of biological entities and their relationships.

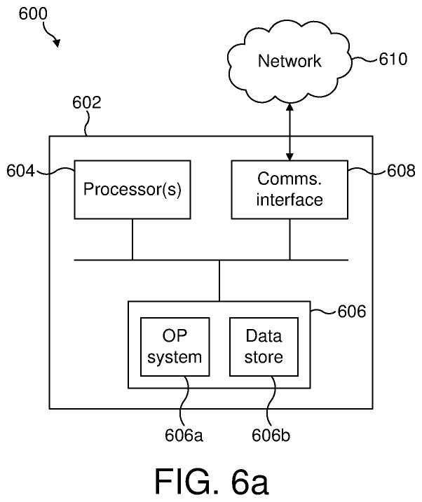

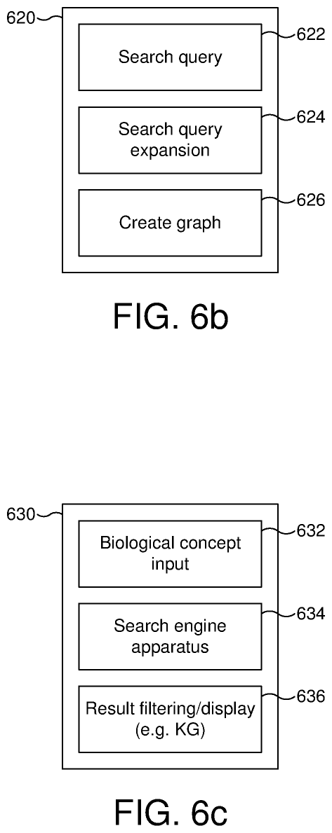

FIG. 6a is a schematic diagram illustrating a computing system 600 including a computing device, server and/or apparatus 602 coupled to a communications network 610 that may be used to implement one or more aspects of the process(es), system(s), method(s) ML model(s) and the like according to the invention and/or implement one or more aspects of the process(es), system(s), method(s) and/or ML model(s) and apparatus as described with reference to FIGS. 1a to 5e and/or 6b and 6c, combinations thereof, modifications thereto, herein described and/or as the application demands. Computing device 602 includes one or more processor unit(s) 604, memory unit 606 and communication interface (CI) 608 in which the one or more processor unit(s) 604 are connected to the memory unit 606 and the communication interface 608. The communications interface 608 may connect the computing device 602 over communication network 610 with one or more databases, corpus of text and/or other processing system(s) or computing device(s)/server(s) and/or client(s) and the like. The memory unit 606 may store one or more program instructions, code or components such as, by way of example only but not limited to, an operating system (OP system) 606a for operating computing device 602 and a data store 606b for storing additional data and/or further program instructions, code and/or components associated with implementing the functionality and/or one or more function(s) or functionality associated with one or more of the method(s) and/or process(es) of the apparatus, module(s), ML model(s), systems(s), mechanisms and/or system(s)/platforms/architectures as described herein and/or as described with reference to at least one of figure(s) la to 5e and 6b and 6c.

As an example, the computing system 602 may be configured to, without limitation, for example interact with the network 610 such that a search query is passed through the network 610 from the client(s) to the search query module. Alternatively, knowledge graph results are passed from the graph creation component to clients via the network 610.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es), modifications thereto, and/or combinations thereof as described herein with reference to any one of FIGS. 1a to 6c

FIG. 6b is a schematic diagram illustrating a system 620 according to the invention. The system comprises a search query module 622, a search query expansion module 624, and a create graph module 626. The search query expansion module 624 attains an expanded search query from the search query module 622 and outputs the validated entities/relationships for the create graph module to generate either a new or updated knowledge graph or graphs. The system 620 and modules/components 622-626 may include the functionality of the method(s), process(es), and/or system(s) associated with the invention as described herein, or as described with reference to FIG. 1a-6c, combinations thereof, modifications thereto and/or as the application demands and the like.

FIG. 6c is a schematic diagram illustrating another system 630 according to the invention. The exemplary system 630 comprises a biological concept input module 632, a search engine apparatus 634, and a result filtering display 636. Here, the biological concept input module receives an input of biological concepts or seed terms. From the biological concepts that are seeded, the search engine apparatus 634 generates a set of entities/relationship and outputs these entities/relationship as knowledge graphs to be displayed by the results filtering display 636. The system 630 and modules/components 632-636 may include the functionality of the method(s), process(es), and/or system(s) associated with the invention as described herein, or as described with reference to FIG. 1a-6c, combinations thereof, modifications thereto and/or as the application demands and the like.

Further aspects of the invention may include one or more apparatus and/or devices that include a communications interface, a memory unit, and a processor unit, the processor unit connected to the communications interface and the memory unit, wherein the processor unit, storage unit, communications interface are configured to perform the system(s), apparatus, method(s) and/or process(es); modifications thereof; combinations thereof; as described herein; and/or as described with reference to FIG. 1a to 6c.

Further aspects of the invention may include a system that includes a user interface configured for receiving one or more entity concepts associated with entities of interest, a search engine apparatus configured to perform or implement the corresponding system(s), apparatus, component(s)/module(s), method(s) and/or process(es); modifications thereof; combinations thereof; as described herein; and/or as described with reference to FIG. 1a to 6c, the search engine apparatus connected to the user interface for receiving the one or more entity concepts. The system may also include a display interface configured for displaying the graph associated with the one or more entity concepts.

Further aspects of the invention may include a system that includes a system that includes a receiver component configured to receive a search query corresponding to entities of interest, the search query including data representative of a first set of entities; a search query expansion component configured to generate an expanded search query based on inputting the received search query to one or more entity expansion process, the expanded search query comprising data representative of a second set of entities and the first set of entities; and a graph creation component configured to create a graph of entities of interest and relationships thereto based on processing the expanded search query with data representative of a corpus of text.

The receiver component, search query expansion component, and the graph creation component may be configured to perform or implement the corresponding system(s), apparatus, component(s)/module(s), method(s) and/or process(es); modifications thereof; combinations thereof; as described herein; and/or as described with reference to figures 1a to 6c.

In the embodiment(s) described above the method(s), apparatus, system(s) and/or computing system/device(s) may be implemented by a server, the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic or semi-automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Application Program-specific Integrated Circuits (ASICs), Application Program-specific Standard Products (ASSPs), System-on-a-chip systems (SOCs). Complex Programmable Logic Devices (CPLDs), etc.

Although illustrated as a single apparatus or system, it is to be understood that the computing device or system may be a distributed system or part of a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface). Furthermore, the systems, apparatus, and/or method(s) as described herein may be distributed or located remotely and accessed via a network or other communication link (e.g. using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “module”, “component” and/or “system” are intended to encompass computer-readable data storage that is configured with computer-executable instructions that cause certain functionality to be performed when executed by a processor. The computer-executable instructions may include a routine, a function, or the like. It is also to be understood that a module, component and/or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims. Although various embodiments have been described above with a certain degree of particularity, or with reference to one or more individual embodiments, those skilled in the art could make numerous alterations to the disclosed embodiments without departing from the spirit or scope of this invention.

1. A computer-implemented method of creating a graph of entities of interest and relationships thereto, the method comprising:
receiving a search query corresponding to entities of interest, the search query comprising data representative of a first set of entities;
generating an expanded search query based on inputting the received search query to one or more entity expansion process(es) the expanded search query comprising data representative of a second set of entities and the first set of entities; and
creating a graph of entities of interest and relationships thereto based on processing the expanded search query with data representative of a corpus of text.

2. The computer-implemented method as claimed in claim 1, wherein generating the expanded search query further comprising:
sending data representative of the received search query to said one or more entity expansion process(es);
receiving data representative of the second set of entities from said one or more entity expansion process(es); and
building an expanded search query corresponding to entities of interest based on a selection of data representative of the second set of entities and the first set of entities in relation to the entities of interest.

3. The computer-implemented method as claimed in claim 1 or 2, wherein generating the expanded search query further comprising iteratively generating the expanded search query by:
sending data representative of a current search query to said one or more entity expansion process(es), wherein, in the first iteration the current search query is the received search query;
receiving data representative of the second set of entities from said one or more entity expansion process(es) based on the current search query; and
building an expanded search query corresponding to entities of interest based on a selection of data representative of the second set of entities and the first set of entities in relation to the entities of interest; and
updating the current search query with the expanded search query in response to performing another iteration.

4. The computer-implemented method as claimed in claim 3, wherein building an expanded search query further comprises:
receiving feedback that one or more of the entities of interest of the expanded search query are valid; and
updating the expanded search query to only include data representative of the valid entities of interest.

5. The computer-implemented method as claimed in any preceding claim, wherein creating the graph by processing the expanded search query further comprising:
performing a search for entities of interest and relationships thereto in the corpus of text based on the expanded search query; and
forming the graph of entities of interest and relationships thereto based on search results output from said search.

6. The computer-implemented method as claimed in any preceding claim, wherein creating the graph by processing the expanded search query further comprises filtering an existing graph of entities of interest and relationships thereto based on the expanded search query, wherein the existing graph of entities of interest and relationships thereto is previously generated based on the corpus of text.

7. The computer-implemented method as claimed in any preceding claim, further comprising:
receiving data representative of an additional set of entities output from one of the entity expansion process(es) configured to retrieve the additional set of entities from a database lookup using data representative of the search query corresponding to entities of interest; and
combining the additional set of entities with the second set of entities.

8. The computer-implemented method as claimed in any preceding claim, further comprising:
receiving data representative of an additional set of entities output from one of the entity expansion process(es) configured to extract entities of interest from or filter an existing graph of entities of interest and relationships thereto based on data representative of the search query; and
combining the additional set of entities with the second set of entities.

9. The computer-implemented method as claimed in any preceding claim, further comprising:
receiving data representative of an additional set of entities output from one of the entity expansion process(es) configured to input data representative of the search query to an ML model trained for predicting or identifying entities of interest and relationships thereto from a corpus of text; and
combining the additional set of entities with the second set of entities.

10. The computer-implemented method as claimed in any preceding claim, further comprising:
receiving data representative of an additional set of entities output from one of the entity expansion process(es) configured to search a corpus of text based on data representative of the search query; and
combining the additional set of entities with the second set of entities.

11. The computer-implemented method as claimed in any preceding claim, further comprising:
receiving data representative of an additional set of entities output from one of the entity expansion process(es) configured to retrieve the additional set of entities from a lexicon dictionary associated with entities; and
combining the additional set of entities with the second set of entities.

12. The computer-implemented method as claimed in any preceding claim, wherein creating a graph of entities of interest and relationships thereto further comprising:
receiving the expanded search query based on a set of entity concepts associated with one or more entities;
retrieving a set of entities and relationships thereto from the corpus of text based on inputting data representative of the expanded search query to a search engine configured for identifying one or more entity(ies) and relationships thereto based on the received expanded search query and the corpus of text; and
generating a graph of entities of interest and relationships thereto using the retrieved set of entities and relationships.

13. The computer-implemented method as claimed in claim 12, wherein retrieving a set of entities and relationships thereto from the corpus of text further comprising:
inputting the expanded search query to a document extraction engine configured for identifying portions of text from the corpus of text associated with the expanded search query;
and outputting one or more identified portions of text from the corpus of text associated with the expanded search query.

14. The computer-implemented method as claimed in any of claim 12 or 13, wherein retrieving a set of entities and relationships thereto from the corpus of text further comprising:
inputting identified portions of text from the corpus of text associated with the expanded search query to a relationship extraction engine configured for identifying or predicting one or more entity(ies) and relationship(s) thereto in relation to the identified portions of text associated with the expanded search query; and
outputting the identified or predicted set of entity(ies) and relationship(s) thereto.

15. The computer-implemented method as claimed in claim 13 or 14, wherein the portions of text comprise a set of relevant documents from the corpus of text that are determined relevant to the entity concepts of the expanded search query.

16. The computer-implemented method as claimed in claim 15, wherein the search engine comprises one or more ML search model(s) configured for identifying, predicting, ranking and/or scoring the plurality of documents associated with the expanded search query for determining the set of relevant documents.

17. The computer-implemented method as claimed in claim 16, wherein the search engine further comprises one or more information retrieval algorithms associated with document frequency and/or document similarity for performing a document search.

18. The computer-implemented method as claimed in any of claims 12 to 17, wherein the relationship extraction engine comprises one or more ML extraction model(s) configured for identifying, predicting, ranking and/or scoring a set of entities and relationships thereto in relation to the identified portions of the set of relevant documents and the expanded search query.

19. The computer-implemented method as claimed in any preceding claim, wherein receiving the search query based on data representative of the first set of entities further comprising receiving data representative of a selected first set of entity concepts associated with one or more entities of interest from a user.

20. The computer-implemented method as claimed in claim 19, wherein generating an expanded search query comprising data representative of a second set of entities and the first set of entities further comprising:
expanding the first set of entity concepts based on an expansion engine configured to expand the first set of entity concepts into data representative of a further relevant set of entity concepts; and
generating an expanded search query based on the first set of entity concepts and/or the further relevant set of entity concepts.

21. The computer-implemented method as claimed in claim 20, wherein expanding the first set of entity concepts further comprising iteratively expanding the first set of entity concepts by:
expanding a current set of entity concepts based on an expansion engine configured to expand the current set of entity concepts into data representative of a further relevant set of entity concepts, wherein in the first iteration the current set of entity concepts is the first set of entity concepts;
receiving feedback that one or more of the entity concepts from the current set of entity concepts and/or further relevant set of entity concepts are valid or of interest;
generating an expanded set of entity concepts based on the validated or of interest entity concepts from the current set of entity concepts and/or further relevant set of entity concepts;
replacing the current set of entity concepts with the expanded set of entity concepts;
iteratively performing the steps of expanding the current set of entity concepts, receiving feedback, and generating an expanded set of entity concepts until a stopping criterion in relation to expanding the current set of entity concepts is reached; and
generating an expanded search query based on the current set of entity concepts.

22. The computer-implemented method as claimed in claim 21, further comprising updating the expansion engine configured to expand a set of entity concepts into further relevant set of entity concepts based on the received feedback of valid or of interest entity concepts.

23. The computer-implemented method as claimed in claim 22, further comprising updating the expansion engine prior to generating the expanded set of entity concepts.

24. The computer-implemented method as claimed in any of claims 20 to 23, wherein the expansion engine comprises one or more entity expansion process(es) from the group of:
an entity expansion process configured to extract additional entities of interest from or filter an existing graph of entities of interest and relationships thereto based on data representative of a set of entity concepts;
an entity expansion process configured to input data representative of a set of entity concepts to an ML model trained for predicting or identifying additional entities of interest and relationships thereto from a corpus of text;
an entity expansion process configured to search for additional entities of interest from a corpus of text based on inputting data representative of a search query associated with a set of entity concepts to a search engine coupled to the corpus of text;
an entity expansion process configured to retrieve additional entities of interest from a lexicon dictionary associated with a set of entity concepts; and
any other entity expansion process configured to retrieve additional entities from a database, dictionary system and/or search engine and the like in relation to a set of entity concepts.

25. The computer-implemented method as claimed in any preceding claim, wherein creating a graph of entities of interest and relationships thereto further comprises:
generating a graph based on the retrieved sets of entities and relationships thereto; and
updating an existing graph associated with the one or more entities of interest based on the generated graph.

26. The computer-implemented method as claimed in any preceding claim, wherein creating a graph further comprises generating a graph based on the retrieved sets of entities and relationships thereto.

27. The computer-implemented method as claimed in any preceding claim, wherein a graph of entities of interest and relationships thereto comprises a graph structure comprising a plurality of nodes based on a set of entities, wherein each node of the graph structure represents an entity and edges between a pair of nodes correspond to a particular relationship between the entities represented by the pair of nodes.

28. The computer-implemented method as claimed in claim 27, generating the graph further comprising:
inferring a relationship edge between a first node and a second node of the graph when a first relationship edge exists from the first node to another node of the graph, and a second relationship edge exists from the another node to the second node; and
inserting an inferred relationship edge between the first node and second node of the graph.

29. The computer-implemented method as claimed in claim 27 or 28, generating the graph further comprising:
inferring, for each node of the plurality of nodes in the graph, a relationship edge between said each node and an other node of the graph when a relationship edge path exists from said each node via one or more further nodes to the other node; and
inserting an inferred relationship edge between said each node and the other node of the graph.

30. The computer-implemented method as claimed in claim 27 or 29, further comprising weighting each relationship edge between each pair of nodes of the graph based on detecting the number of common relationships between the entities of said each pair of nodes from the set of entities and relationships.

31. The computer-implemented method as claimed in any preceding claim, wherein retrieving a set of entities and relationships thereto from the corpus of text using one or more ML extraction model(s) further comprising:
generating predictions based on the expanded search query using one or more machine learning, ML, model(s) configured for predicting from the corpus of text a set of entity pairs and relationships associated with a set of entities associated with the search query, each predicted entity pair comprising an entity of a first type and an entity of a second type having an associated relationship therebetween identified from the corpus of text; and
outputting the set of entity pairs and relationships as the set of entities and relationships.

32. The computer-implemented method as claimed in any preceding claim, wherein the data representative of the graph is used as input labelled training datasets for training one or more ML model(s) associated with predicting or classifying objective problems and/or processes in the field of: biology, biochemistry, chemistry, medicine, chem(o)informatics, bioinformatics, pharmacology, and any other field relevant to diagnostic, treatment, and/or drug discovery and the like.

33. The computer-implemented method as claimed in any preceding claim, wherein an entity comprises entity data associated with an entity type from at least the group of: gene; disease; compound/drug; protein; chemical; organ; biology; biological part; or any other entity type associated with bioinformatics, chem(o)informatics, biology, biochemistry, chemistry, medicine, pharmacology, and/or any other field relevant to diagnostic, treatment, and/or drug discovery and the like.

34. The computer-implemented method as claimed in any preceding claim, wherein an entity concept is data representative of entity information and/or entities from one or more fields or domains from the group of: biology, biochemistry, chemistry, medicine, chem(o)informatics, bioinformatics, pharmacology, and/or any other field relevant to diagnostic, treatment, and/or drug discovery and the like.

35. A search engine apparatus for searching and filtering entity results for entities of interest from an corpus of text, the search engine apparatus comprising:
an input component configured to receive a search query based on set of entity concepts associated with one or more entities;
an expansion component configured to expand the received search query into an expanded search query comprising at least the set of entity concepts and/or further relevant entity concepts associated with the set of entity concepts;
a search processor component configured to retrieve a set of entities and relationships thereto from the corpus of text based on inputting the expanded search query to a search engine configured for identifying and/or predicting one or more entity(ies) and relationship(s) thereto based on the expanded search query and the corpus of text;
an entity result filtering component configured generate a graph using the retrieved set of entities and relationships thereto.

36. The search engine apparatus as claimed in claim 35, wherein the input component, expansion component, the search processor component and/or the entity result filtering component are configured to implement the computer-implemented method according to any of claims 1 to 34.

37. An apparatus comprising a processor unit, a memory unit and a communication interface, the processor unit connected to the memory unit and the communication unit, wherein the apparatus is configured to implement the computer-implemented method according to any of claims 1 to 34.

38. A system comprising:
a user interface configured for receiving one or more entity concepts associated with entities of interest;
a search engine apparatus configured according to any of claims 35 to 36 connected to the user interface for receiving the one or more entity concepts; and
a display interface configured for displaying the graph associated with the one or more entity concepts.

39. A system comprising:
a receiver component configured to receive a search query corresponding to entities of interest, the search query comprising data representative of a first set of entities;
a search query expansion component configured to generate an expanded search query based on inputting the received search query to one or more entity expansion process, the expanded search query comprising data representative of a second set of entities and the first set of entities; and
a graph creation component configured to create a graph of entities of interest and relationships thereto based on processing the expanded search query with data representative of a corpus of text.

40. The system as claimed in claim 39, wherein the receiver component, search query expansion component, and the graph creation component are configured to implement the computer-implemented method according to any of claims 1 to 34.

41. A computer-readable medium comprising code or computer instructions stored thereon, which when executed by a processor unit, causes the processor unit to perform the computer-implemented method according to any one of claims 1 to 34.

42. The computer-implemented invention, search engine apparatus, system as claimed in any preceding claim, wherein the corpus of text comprises a large-scale document repository including a plurality of documents associated with a plurality of entity concepts and/or entities of interest and/or entities of relevance.
